(19) United States
(12) Patent Application Publication
(10) Pub. No.: US 2026/0283445 A1
(43) Pub. Date: Sep. 24, 2026

(54) ENDOSCOPIC DEVICES AND METHODS AND RELATED IMAGING PROCESSING TECHNIQUES

(71) Applicant: Decode Bone LLC, Milpitas, CA (US)

(72) Inventor: Amrish J. Walke, Milpitas, CA (US)

(21) Appl. No.: 19/577,294

(22) Filed: Mar. 24, 2026

Related U.S. Application Data
(60) Provisional application No. 63/776,549, filed on Mar. 24, 2025.

(51) Int. Cl. A61B 1/00; A61B 1/303; A61B 34/20; A61F 6/18

(52) U.S. Cl. CPC A61B 1/000096; A61B 1/0005; A61B 1/303; A61B 34/20; A61F 6/18; A61B 2034/2065

(57) ABSTRACT

A system and method for extracting clinically relevant information from endoscopic videos and images are provided. The system processes imaging data obtained from an endoscope to identify anatomical structures and pathological features within an anatomical cavity. Image data are analyzed to determine quantitative parameters including size, shape, contour, and spatial relationships of anatomy and pathology. The system generates measurement data and anatomical representations that may be used to support diagnostic and therapeutic procedures. In some embodiments, artificial intelligence models are applied to perform feature extraction, segmentation, and analysis of endoscopic imaging outputs. The extracted information is used to guide device placement, navigation, and treatment within the anatomical cavity. The system may further provide outputs indicative of uterine conditions, including fibroids and abnormal bleeding, to assist clinical decision-making and improve procedural accuracy.

### CROSS-REFERENCE TO RELATED APPLICATIONS

This application claims the priority benefit of U.S. Provisional Patent Application Ser. No. 63/776,549, filed March 24, 2025, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### BACKGROUND

Endoscopic procedures generate video and photographic data that document anatomical features and procedural observations. In many settings, such data are summarized in brief narrative or tabular reports, such as the one- or two-page reports conventionally produced for hysteroscopy procedures. These reports often provide only high-level findings and may not include the full range of visual information available during the procedure. For instance, quantitative details, such as uterine cavity dimensions, and qualitative features, such as cavity contours or structural characteristics observed during hysteroscopy, are typically not captured in existing reporting formats. Additionally, conventional hysteroscopic systems primarily serve as visualization tools and do not incorporate functionality to assist the user in acquiring, analyzing, or interpreting procedural data.

### SUMMARY

In some aspects, the techniques described herein relate to a system for imaging an interior of an anatomical cavity, the system includes: an endoscope configured for an insertion into the anatomical cavity; a display device; and a controller receiving an imaging output of the endoscope, wherein the controller includes a processor and a memory storing instructions and one or more trained artificial intelligence models, wherein the instructions, when executed by the processor, cause the controller to: process the imaging output of the endoscope using the one or more trained artificial intelligence models to generate computed information of the anatomical cavity, wherein the computed information includes one or more of: size information of the anatomical cavity, shape information of the anatomical cavity, and identification of pathology; and display a graphical overlay on the display device, the graphical overlay displaying the computed information of the anatomical cavity and the imaging output of the endoscope.

In some aspects, the techniques described herein relate to a method of performing a diagnostic or therapeutic procedure on an anatomical cavity including: inserting an endoscope into the anatomical cavity; imaging a wall of the anatomical cavity using the endoscope; determining one or more contours of the wall of the anatomical cavity; and using the one or more contours to guide placement and/or function of working device and/or the endoscope within the anatomical cavity.

### BRIEF DESCRIPTION OF THE FIGURES

The foregoing is a summary, and thus, necessarily limited in detail. The above-mentioned aspects, as well as other aspects, features, and advantages of the present technology are described below in connection with various embodiments, with reference made to the accompanying drawings.

FIGS. 1A-1G show the steps of a method of performing a hysteroscopy on a patient, according to some embodiments.

FIG. 1H shows a reconstructed view of the endometrial cavity where in one or more desired measurements of the endometrial cavity have been performed by the system, according to some embodiments.

FIG. 1I and FIG. 1J show two steps of a method of introducing a device into the fallopian tubes or the cornua of a patient, according to some embodiments.

FIG. 1K shows an embodiment of cornual region mapping, according to some embodiments.

FIGS. 1L-1N show the steps of a method of navigating a hysteroscope through the cervix, according to some embodiments.

FIG. 2A shows an embodiment of the system where the uterine cavity width has been calculated, according to some embodiments.

FIG. 2B shows an embodiment of the system wherein the deployment of an endometrial ablation device has been predicted by the system, according to some embodiments.

FIG. 2C shows an embodiment of the system, wherein the location of an intrauterine device (IUD) has been predicted and/or calculated by the system, according to some embodiments.

FIG. 2D shows another embodiment of the system, wherein the location of an intrauterine device IUD has been predicted and/or calculated by the system, according to some embodiments.

FIG. 2E shows an embodiment of the system for measuring intrauterine pathology. In this embodiment, a polyp has been detected by the system, and the dimensions of the polyp have been measured by the system, according to some embodiments.

FIG. 2F shows an embodiment of the system where in one or more adhesions have been analyzed by the system, according to some embodiments.

FIG. 2G shows a step of a method of accessing the cornua or the fallopian tube lumen using an embodiment of the present invention, according to some embodiments.

FIG. 2H shows an embodiment of the system for directing the user to sites for injecting local anesthesia or other medications, according to some embodiments.

FIGS. 2I-2L show various steps of method embodiments of predicting the performance of endometrial ablation inside a uterine cavity, according to some embodiments.

FIGS. 2M and 2N show various method embodiments of selecting an IUD for the uterine cavity based on cavity size and other anatomical characteristics or parameters, according to some embodiments.

FIG. 2O shows a device being used to treat or a diagnose a portion of the uterine cavity wherein the insertion, deployment, movements, or actions of the device are based on information obtained prior to the insertion of this device, according to some embodiments.

FIGS. 2P-2S show various embodiments of methods and devices to treat pathology located within a wall of an anatomical cavity, according to some embodiments.

FIGS. 3A-3C show embodiments of visual outputs of some embodiments of the present invention wherein one or more pathologies have been marked, according to some embodiments.

FIGS. 3D and 3E show images of the uterine cavity obtained by some embodiments before and after septoplasty, according to some embodiments.

FIGS. 3F and 3G and show images of the uterine cavity obtained by some embodiments before and after myomectomies, according to some embodiments.

FIGS. 3H and 3I show images of the uterine cavity at two time points showing the growth of a polyp, according to some embodiments.

FIG. 3J shows an embodiment wherein regions of the uterine cavity are magnified and mapped to zones of an output image, according to some embodiments.

FIG. 3K shows an embodiment wherein the curved uterine surface has been projected to a 2D surface, according to some embodiments.

FIG. 3L and FIG. 3M show embodiments of an output image of the present invention showing various orientations, sizes, distances, angles, according to some embodiments.

FIG. 3N shows an embodiment of a hysteroscopy report produced by some embodiments of the present invention, according to some embodiments.

FIGS. 3O-3Q show examples of two-dimensional visual/graphical representation of the uterine anatomy and/or pathology that may be created by some device and method embodiments, according to some embodiments.

FIGS. 4A-4G show various method embodiments of the present invention, according to some embodiments.

FIG. 4H shows an example of a method that uses multi-modal data and data fusion techniques, according to some embodiments.

FIG. 4I shows an example of a method of training a Large Language Model (LLM) and using such trained LLMs for clinical applications, according to some embodiments.

FIGS. 5A-5E show various components of a system for performing diagnostic as well as therapeutic hysteroscopy, according to some embodiments.

FIGS. 6A-6C show examples of flexible digital endoscopes, according to some embodiments.

FIGS. 6D-6G show the steps of an example of a method of using a flexible digital endoscope, according to some embodiments.

FIG. 7 illustrates a box diagram of an example embodiment of a system for imaging and navigating within an anatomical cavity, including components that support controlled steering of a hysteroscope, according to some embodiments.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The present disclosure relates to systems and/or methods for extracting clinically useful information from endoscopic videos and/or images and/or using such information for diagnostic and/or therapeutic applications. There is a need for new and/or improved systems and/or methods that go beyond conventional visualization-only endoscopic systems by enabling quantitative and/or structured analysis of anatomical regions observed during procedures. In many existing workflows, endoscopic observations are subjectively interpreted and/or summarized in limited reports, which may omit detailed spatial and/or geometric and/or pathological information. Such limitations may reduce the ability to accurately diagnose conditions and/or plan interventions and/or standardize clinical decision making across users and/or institutions. Accordingly, there is a need for systems and/or methods that automatically derive clinically meaningful information from endoscopic imaging data in a consistent and/or reproducible manner. The present disclosure addresses these needs by providing systems and/or methods that process endoscopic imaging outputs to generate structured anatomical and/or pathological information.

In particular, there is a need for systems, devices, and/or methods that obtain anatomy and/or pathology-related clinical information, including size measurements and/or shape characteristics and/or spatial relationships and/or structural features, to guide diagnosis and/or therapy for uterine conditions such as fibroids and/or heavy menstrual bleeding. Conventional hysteroscopic systems primarily provide visual access to the uterine cavity without offering tools for quantitative measurement and/or predictive analysis. As a result, clinicians may rely on subjective estimation when assessing cavity dimensions and/or pathology characteristics. This may lead to variability in diagnosis and/or suboptimal device selection and/or incomplete treatment. The disclosed embodiments provide mechanisms for extracting quantitative anatomical data and/or pathology-related metrics from imaging outputs, thereby enabling improved procedural planning and/or therapeutic precision. The systems and/or methods described herein may reduce reliance on subjective interpretation and/or improve consistency across clinical users and/or environments.

The present disclosure includes systems based on endoscopes for imaging the interior of anatomical cavities and/or lumens and/or surgically created cavities or passages. Such anatomical cavities may include, without limitation, uterine cavities and/or fallopian tubes and/or vaginal canals and/or urinary tract lumens and/or gastrointestinal lumens and/or vascular structures and/or other internal anatomical regions accessible via endoscopic instruments. An endoscope may include one or more imaging sensors configured to capture video and/or still images of an interior surface of an anatomical cavity. The imaging data generated by the endoscope may be transmitted to a controller and/or processing system for further analysis. In some embodiments, the endoscope may include additional components such as illumination sources and/or optical elements and/or positioning sensors and/or orientation sensors to facilitate imaging and/or navigation within the anatomical cavity. Such configurations enable acquisition of imaging data suitable for downstream computational analysis and/or clinical interpretation.

In some embodiments, the present disclosure includes systems in which a controller receives and/or processes imaging outputs of an endoscope. The controller may include one or more processors and/or one or more memory components storing executable instructions and/or one or more trained artificial intelligence models. The controller may process the imaging outputs to determine features of the anatomical cavity, including contours of cavity walls and/or anatomical landmarks and/or regions of interest. The processing may include application of computer vision techniques such as edge detection and/or segmentation and/or contour extraction and/or feature tracking and/or geometric analysis. In some embodiments, the processing may further include application of artificial intelligence models, including machine learning and/or deep learning models, trained to identify anatomical structures and/or classify tissue types and/or detect pathological features. Such processing enables automated and/or semi-automated interpretation of endoscopic imaging data.

Such processing may determine contours of walls of anatomical cavities and/or generate representations of those contours in one or more coordinate systems. The system may analyze these contours to derive geometric parameters, including distances and/or angles and/or curvature and/or relative spatial relationships between anatomical features. These parameters may be used to generate computed information of the anatomical cavity, including size parameters and/or shape parameters and/or structural characteristics. In some embodiments, the system may further identify and/or characterize pathologies and/or abnormalities, including fibroids and/or polyps and/or adhesions and/or other tissue irregularities, based on the processed imaging data. The identification of pathology may include determining boundaries and/or dimensions and/or spatial location of the pathology relative to surrounding anatomical structures. Such computed information may be stored and/or displayed and/or used for subsequent diagnostic and/or therapeutic processes.

The computed information generated by the system may be used to guide imaging of the anatomical cavity wall and/or improve completeness of data acquisition. For example, the system may determine whether certain regions of the anatomical cavity have not been adequately imaged and/or may provide guidance to a user to reposition the endoscope to capture additional views. The system may also provide real-time and/or near real-time feedback during imaging, including visual overlays indicating target regions and/or alignment markers and/or recommended trajectories for advancing the endoscope. Such guidance may improve imaging coverage and/or reduce the likelihood of missing clinically relevant features. In some embodiments, the system may dynamically update guidance based on changes in the imaging output and/or detected anatomical features. This enables adaptive imaging workflows that respond to intra-procedural conditions.

The present disclosure further includes systems and/or methods of tracking and/or using working devices that are introduced into anatomical cavities. A working device may include any device configured to perform a diagnostic and/or therapeutic function within the anatomical cavity, including, but not limited to, ablation devices and/or biopsy devices and/or cutting tools and/or injection devices and/or implant delivery systems. In some embodiments, the system tracks the position and/or orientation and/or movement of the working device relative to the anatomical cavity using imaging data and/or sensor data and/or a combination thereof. The tracking information may be integrated with the computed anatomical information to provide context-aware guidance during device manipulation. Such integration enables coordinated interaction between imaging and/or intervention.

In some embodiments, the working parameters of such working devices are determined using the computed information of the anatomical cavity. For example, the system may determine appropriate insertion depth and/or orientation and/or deployment configuration and/or operational settings of a working device based on cavity dimensions and/or shape characteristics and/or identified pathology. The system may further calculate predicted outcomes of device deployment, such as coverage of a treatment region and/or interaction with surrounding tissue. Such determinations may be used to assist the user in performing procedures more accurately and/or safely. In some embodiments, the system may automatically adjust and/or recommend adjustments to device parameters based on the computed anatomical information. This may reduce procedural variability and/or improve treatment outcomes.

The present disclosure also includes systems and/or methods of displaying clinically relevant information to a user. The system may generate one or more visual outputs, including two-dimensional and/or three-dimensional representations of the anatomical cavity. Such representations may be reconstructed from imaging data using geometric modeling techniques and/or multi-view reconstruction and/or depth estimation methods. The representations may provide a comprehensive view of the anatomical cavity, including spatial relationships between different regions and/or features. In some embodiments, such visualizations may be used before and/or during and/or after a procedure to support clinical workflows.

In some embodiments, the visual representations may include additional system-generated information such as annotations and/or navigation guides and/or size measurements and/or shape parameters and/or identified pathology. These overlays may be displayed in conjunction with real-time and/or recorded endoscopic images on a display device. The overlays may include graphical elements such as markers and/or contours and/or labels and/or indicators that assist the user in interpreting the imaging data and/or performing procedures. The system may also present numerical data and/or alerts and/or recommendations derived from the computed information. Such integrated visualization enables improved understanding of anatomical and/or pathological conditions.

The systems and methods described herein provide technical solutions that improve the extraction and/or analysis and/or utilization of clinically relevant information from endoscopic imaging data. For example, conventional endoscopic systems are primarily designed to provide visualization of internal anatomical structures and/or cavities, but they lack the ability to extract and/or present quantitative and/or structured clinical information from the imaging data. In many clinical workflows, a user observes endoscopic video and/or images and subjectively interprets anatomical features and/or pathology, which may lead to variability in diagnosis and/or treatment planning. Existing systems typically do not provide automated measurement of anatomical dimensions and/or shape characteristics and/or spatial relationships, thereby limiting the precision of clinical assessments. Additionally, conventional reporting mechanisms often summarize findings in a limited and/or qualitative manner, omitting detailed geometric and/or structural information that may be relevant to clinical decision making. As a result, clinicians may face challenges in consistently identifying pathology and/or accurately characterizing anatomical conditions. Accordingly, there exists a technical problem of insufficient extraction and/or utilization of clinically meaningful information from endoscopic imaging outputs.

Another technical problem arises in the inability of conventional systems to guide imaging acquisition and/or ensure comprehensive visualization of anatomical regions. Endoscopic procedures rely on the user to manually navigate the endoscope and/or determine whether sufficient imaging coverage has been obtained. However, anatomical cavities often include complex geometries, occluded regions, and/or variable orientations that may result in incomplete imaging. Conventional systems do not provide real-time feedback indicating whether portions of the anatomical cavity have been inadequately imaged or require additional visualization. This may lead to missed pathology and/or incomplete diagnostic information. Furthermore, users may have difficulty aligning the endoscope with optimal viewing angles and/or trajectories, particularly in anatomically constrained or curved regions. Thus, there is a technical problem of limited guidance for imaging completeness and/or optimal navigation during endoscopic procedures.

A further technical problem relates to the lack of integration between imaging data and the operation of working devices introduced into anatomical cavities. In existing systems, working devices such as ablation tools and/or biopsy instruments are typically manipulated without direct computational assistance based on anatomical measurements derived from imaging data. This may result in suboptimal device placement and/or inaccurate targeting of pathology and/or increased risk of unintended tissue interaction. Conventional systems do not provide real-time tracking of working devices relative to anatomical landmarks and/or computed cavity geometry. Additionally, the absence of automated determination of working parameters, such as insertion depth and/or orientation and/or deployment configuration, may reduce procedural accuracy and/or safety. Accordingly, there is a technical problem of insufficient coordination between imaging analysis and device operation within anatomical cavities.

The system provides technical solutions to these problems by implementing a system that processes endoscopic imaging outputs using computational techniques and/or trained artificial intelligence models to generate computed information of an anatomical cavity. The system analyzes imaging data to determine contours of cavity walls and/or identify anatomical landmarks and/or detect pathology. Based on these contours, the system derives quantitative parameters including size and/or shape and/or spatial relationships of anatomical features. This computed information is used to generate structured outputs that provide objective and/or reproducible clinical data. By transforming raw imaging data into quantitative and/or analyzable information, the system improves diagnostic accuracy and/or reduces reliance on subjective interpretation.

The system further provides a technical solution for guiding imaging acquisition by generating real-time feedback based on analysis of imaging outputs. The system determines whether regions of the anatomical cavity have been adequately imaged and/or identifies areas requiring additional visualization. The system may display graphical overlays including alignment markers and/or target regions and/or recommended trajectories to guide repositioning of the endoscope. Such overlays may be dynamically updated based on changes in the imaging output and/or detected anatomical features. This enables adaptive imaging workflows that improve coverage and/or reduce the likelihood of missed pathology. By providing real-time guidance and/or feedback, the system enhances the efficiency and/or completeness of endoscopic imaging procedures.

The system also provides a technical solution for integrating imaging-derived information with the operation of working devices. The system tracks the position and/or orientation and/or movement of a working device within the anatomical cavity using imaging data and/or sensor data. The system uses computed anatomical information to determine working parameters of the working device, including insertion depth and/or orientation and/or deployment configuration. In some embodiments, the system generates guidance information to assist in positioning and/or operating the working device relative to anatomical landmarks and/or pathology. The system may further calculate predicted outcomes of device operation and/or identify potential risks associated with device placement. This integration of imaging analysis and/or device control improves procedural precision and/or safety.

Additionally, the system provides technical solutions for presenting clinically relevant information in an intuitive and/or actionable format. The system generates two-dimensional and/or three-dimensional representations of the anatomical cavity based on processed imaging data. These representations may include annotations and/or measurements and/or navigation guides and/or pathology indicators that assist the user in interpreting the anatomical structure. The system may display such information as graphical overlays on real-time imaging outputs and/or as reconstructed visual models. This approach enhances user understanding of spatial relationships and/or anatomical features and/or procedural context. By combining visualization and/or quantitative analysis and/or guidance, the system improves clinical decision making and/or procedural outcomes.

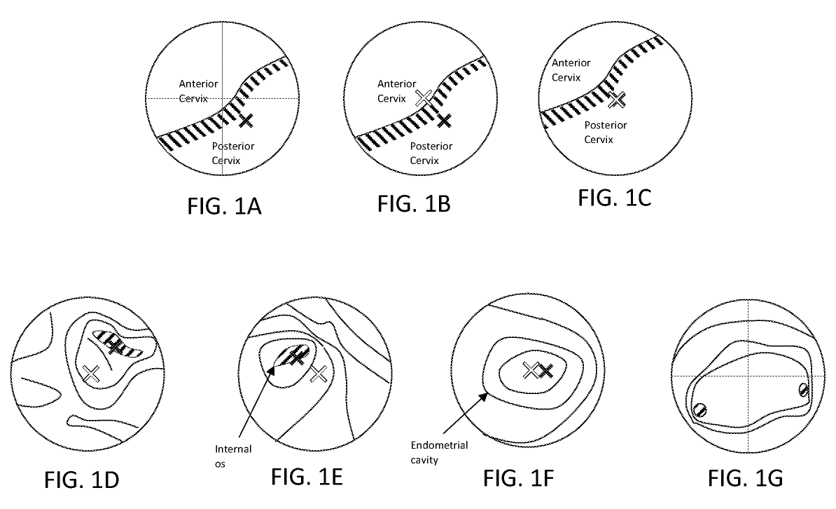

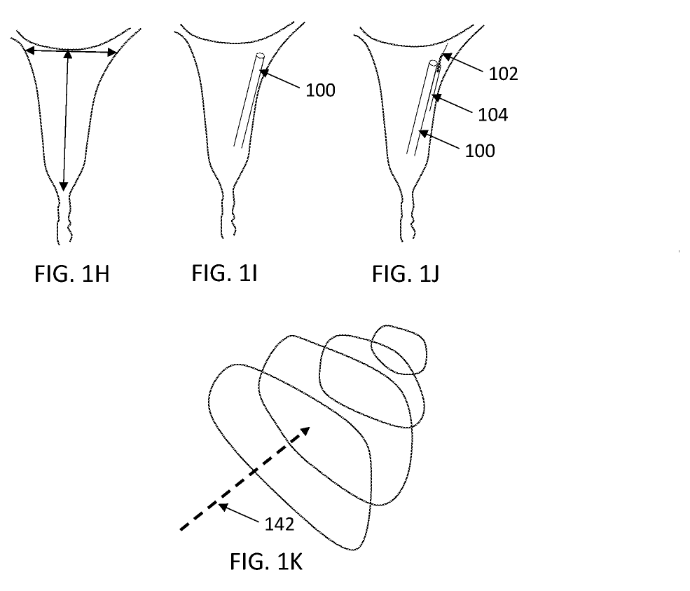

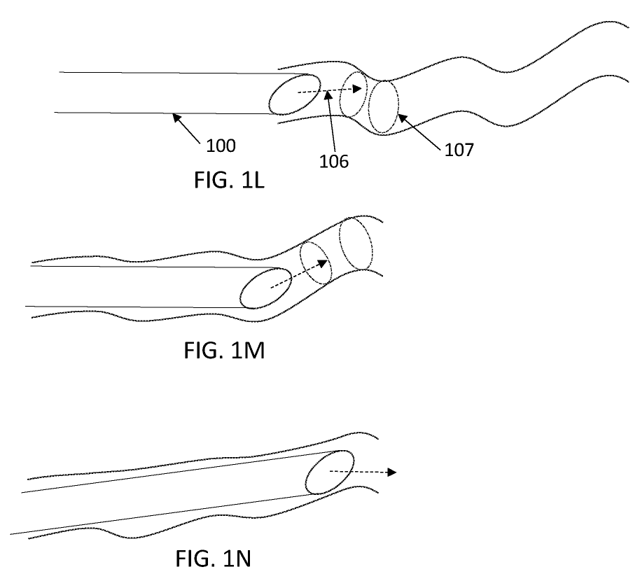

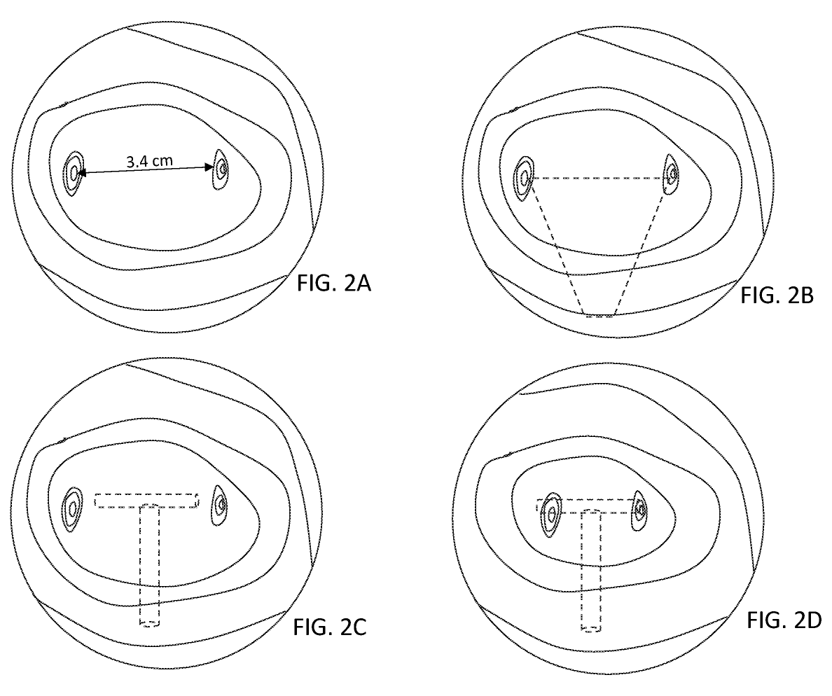

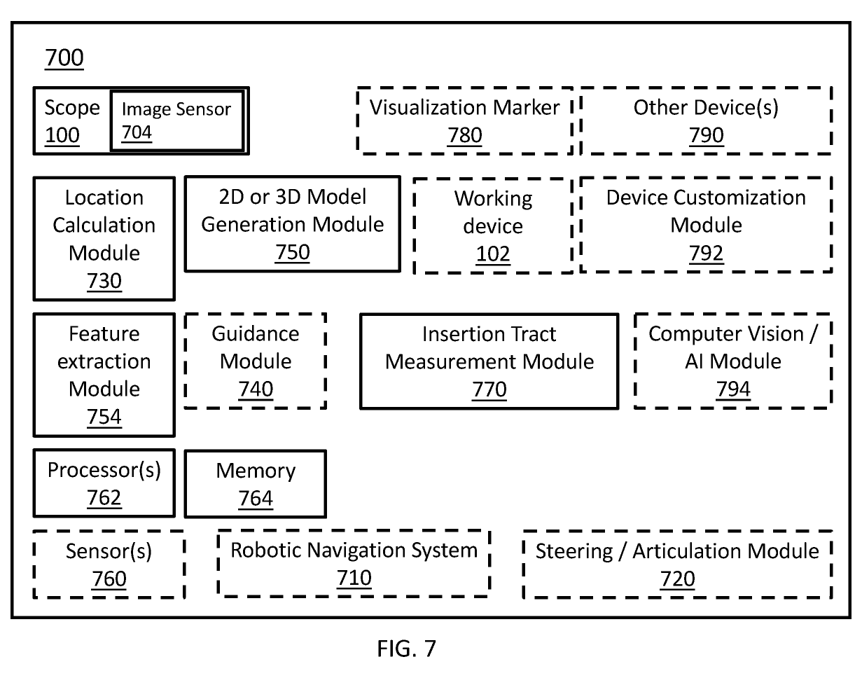

FIGS. 1A-1G show the steps of a method of performing a hysteroscopy on a patient. In the step of FIG. 1A, a hysteroscope is inserted in the vagina and positioned in front of the external orifice. The center of the hysteroscope 100 field of view is shown by the center of the crosshairs. FIG. 1A shows a view of the external opening of the cervix (os) showing the anterior cervix and the posterior cervix. In this embodiment, the system analyzes the contours of the anatomy and determines an optimized location for the center of the hysteroscope 100 field of view (FOV) for cervical insertion. As shown in FIG. 1A, the hysteroscope is oriented to the left upper region of the optimized location (e.g., desired, target, etc.). As shown in FIG. 7, an optimized trajectory may be calculated, using a trajectory calculation module 730, based on contours of the cervical opening. For example, in some embodiments, an optimized trajectory may be determined by analyzing image data (e.g., received from an image sensor 704 of hysteroscope 100) representing the contours of the cervical opening during a hysteroscopy procedure. The system 700 may process endoscopic images to identify boundary features of the cervical os and generate a contour representation. Based on the contour representation, the system 700 may compute a location of interest, such as a central position, alignment point, or path, using one or more geometric or image processing operations. In some embodiments, system 700 may compute the optimized trajectory where the axis of hysteroscope 100 is parallel to the axis of the anatomical region (e.g., cervical canal) at the distal end of hysteroscope 100 and the distal end of hysteroscope 100 lies at the center of the anatomical region (e.g., cervical canal). This trajectory prevents inadvertent scraping or perforation of the anatomical region and improves procedure tolerability in office procedures. The resulting trajectory may reflect a position that facilitates subsequent navigation, measurement, and/or device placement within the uterine cavity. In some embodiments, hysteroscope 100 may be moved and navigated to the optimized trajectory as shown on FIG. 1C. FIG. 1B shows an alternate view of the scope video display where the center of the hysteroscopic FOV is shown by a white “X”. As shown in FIG. 1C, the hysteroscope is reoriented and advanced into the cervix. FIG. 1D shows a view of the cervical canal from the hysteroscope. Similar to FIGS. 1A and 1B, the system shows the optimized trajectory of the center of the hysteroscopic image with a black X (i.e., a system-generated annotation, for example using optional guidance module 740). Such feedback is especially useful when a hysteroscope 100 with a non-zero viewing angle is inserted into the anatomy. Hysteroscope 100 may be advanced using the black X as a guide through the cervix. FIG. 1E shows a view of the distal region of a cervix showing an updated location of the black X, which can be used as a guide to advance hysteroscope 100 further through the cervix. FIG. 1F shows a view of the distal region of the cervix where the uterine (endometrial) cavity is visible. The system further guides the user using the black X for proper insertion into the uterine cavity. In some embodiments, hysteroscope 100 may be advanced into the uterine cavity. FIG. 1G shows a panoramic view of the uterine cavity where both cornua are visible. The hysteroscope 100 may be navigated through the uterine cavity to image the entire uterine cavity.

In some embodiments, as shown in FIG. 7, guidance may be provided through a user interface of optional guidance module 740. For example, the system 700 may display a graphical indicator, such as the black “X” shown in FIG. 1F, at a position corresponding to a recommended insertion direction or entry point into the uterine cavity. As the hysteroscope 100 advances or the hysteroscopic field of view changes, the system 700 may update the position of the indicator so that the hysteroscope 100 can be aligned with the recommended trajectory. In some embodiments, the system 700 may further present, additionally or alternatively, other user interface elements, such as visual cues, overlays, alignment markers, or other user interface elements that assist in orienting or advancing the hysteroscope 100 through the cervical canal and into the uterine cavity. Such guidance may be based on real-time image analysis, orientation data, or other information available to the system 700.

FIG. 1H shows a reconstructed view of the endometrial cavity where in one or more desired measurements of the endometrial cavity have been performed by the system. In some embodiments, this view may be obtained by processing hysteroscopic videos and/or images and creating a 2D profile of the uterine cavity, as shown in FIG. 7 as 2D or 3D model generation module 750. FIG. 1H shows the cervical canal and the uterine cavity and the cornual openings. The system has also calculated uterine cavity width at the level of the mid fundus, and uterine cavity length which has been calculated from the mid fundus to the internal os. Such calculated uterine cavity dimensions, outlines, profiles, etc., may be used for guiding or performing any suitable uterine cavity interventions, some examples of which are disclosed herein.

In some embodiments, the system 700 may analyze hysteroscopic image or video data to extract features corresponding to anatomical boundaries of the uterine cavity. The system 700 may use these extracted features to generate a two-dimensional (2D) profile or reconstructed view of the uterine cavity, such as the view shown in FIGS. 1H and in 7 as 2D or 3D model generation module 750. For example, contours associated with the cervical canal, the uterine cavity walls, and/or the cornual openings may be identified from one or more frames of the hysteroscopic video. Using these contour representations, the system 700 may compute distances between selected anatomical landmarks, for example using 2D or 3D model generation module 750.

By way of illustration, the system 700 may determine a uterine cavity width by identifying contour points located on opposite lateral walls at approximately the level of the mid fundus and calculating the distance between those points. Similarly, a uterine cavity length may be calculated by identifying a location corresponding to the mid fundus and a location corresponding to the internal os and computing a distance between those locations along the reconstructed profile. In some embodiments, the internal os may be determined by determining the boundary of endometrial surface and the cervical surface. In some embodiments, any suitable geometric, image processing, or feature extraction techniques (e.g., using feature extraction module 754) may be used to determine boundaries or reference points for such measurements. For example, such information may be used in guiding or performing uterine cavity interventions, including those described elsewhere herein. By way of non-limiting example, the system 700 may employ one or more geometric operations (e.g.,, distance or centroid calculations), image processing operations (e.g.,, edge detection, thresholding, filtering, or segmentation), or feature extraction operations (e.g.,, contour extraction, landmark identification, or feature tracking) to determine anatomical boundaries or reference points used for the measurements. The calculated cavity dimensions, outlines, profiles, or other quantitative information may then be presented within the reconstructed view.

FIGS. 1I and 1J show two steps of a method of introducing a device into the fallopian tubes or the cornua of a patient. In FIG. 1I, a hysteroscope 100 is inserted into the uterine cavity. One or more methods disclosed herein may be used to insert hysteroscope 100 inside the uterine cavity. In FIG. 1J, a fallopian tube device 102 is introduced into the cornua or into the fallopian tubes through a channel or other guide located on or near hysteroscope 100. As shown in FIG. 7, the system 700 may analyze the anatomy around the cornua and creates a three-dimensional (3D) map of the cornual anatomy, for example using 2D or 3D model generation module 750. Fallopian tube device 102 may be guided using a method like that shown in FIGS. 1A to 1G. Various markers or other aids may be displayed to guide the introduction of the fallopian tube device 102 into the cornua or the fallopian tubes. Examples of fallopian tube device 102 include, but are not limited to: cannulas and other hollow devices, tubal lumen blocking devices, endoscopes, diagnostic devices, etc.

For example, in some embodiments, the system 700 may analyze hysteroscopic image and/or video data obtained when the hysteroscope is positioned near the cornua. From these data, the system may identify anatomical boundaries or features associated with the cornual region, such as tissue contours, lumen openings, or other visible structural transitions. Using these features, the system may construct a 3D representation, such as a depth map, surface map, or volumetric estimate, of the cornual anatomy, for example, using 2D or 3D model generation module 750.

The 3D map may be generated using any suitable technique, such as combining information from multiple image frames, estimating depth from observed motions of the hysteroscope, or integrating geometric relationships derived from identified anatomical contours. For example, the system may track the appearance of the cornual structures across different frames as the hysteroscope moves and may infer relative spatial positions of tissue surfaces based on the change in perspective. In some embodiments, the system may also apply image processing or feature extraction methods (e.g., using feature extraction module 754) to detect edges, contours, or lumen boundaries that define the shape of the cornual region. The resulting 3D map may be displayed or used by the system to provide visual markers, alignment indicators, or other guidance aids, for example using guidance module 740, that assist in introducing fallopian tube device 102 into the cornua or fallopian tubes.

FIG. 1K shows an embodiment of cornual region mapping. The 3D contours of the cornual opening have been displayed as multiple loops on a user interface. The system may also display an optimized or target path of insertion of fallopian tube device 102 and/or other devices into the cornua or into the fallopian tubes. Such 3D contours may be obtained for the walls of at least a portion of the uterine cavity or the entire uterine cavity. Such fallopian tube devices 102 may be navigated to the distal fallopian tube for tissue or cell sampling, delivering drugs or biologics, etc.

FIGS. 1L - 1N show the steps of a method of navigating a hysteroscope through the cervix. In FIG. 1L, hysteroscope 100 is introduced into the cervical canal. As shown, the viewing angle of hysteroscope 100 is at an angle to the axis of the scope. Users often incorrectly advance such hysteroscopes, thereby causing trauma to the cervical walls which in turn causes pain or bleeding. In FIG. 1L, the system may calculate an optimized path 106 for advancement of hysteroscope 100. Path 106 may be displayed or otherwise communicated to a user, for example on a user interface. For example, a visual marker of the optimized location of the center of the hysteroscopic image may be displayed to the user. The orientation of hysteroscope 100 may be adjusted accordingly, and the hysteroscope 100 advanced through the cervical canal. In some embodiments, the orientation of hysteroscope 100 relative to the cervical canal may be determined by generating wall contours 107 of the cervical canal as seen by the hysteroscopic view. Similarly, as shown in FIG. 1M, the system may recalculate the optimized location of the center of the hysteroscopic image. The information may be used to readjust the orientation of hysteroscope 100 and advance hysteroscope 100 further through the anatomy. In this step, the orientation of hysteroscope 100 may be adjusted to a more superior orientation. Similarly, as shown in FIG. 1N, the system may recalculate the optimized orientation of the center of the hysteroscopic image. The orientation of the distal end of hysteroscope 100 may be adjusted to a more inferior orientation. Hysteroscope 100 may thus be navigated through anatomy without causing significant pain or bleeding to the patient. This method also prevents (or reduces the likelihood) hysteroscope 100 from getting stuck in tight corners or sharp bends (e.g., in retroflexed uteri) or pockets of the anatomy. This method is especially useful for hysteroscopes 100 with an angled view. In such angled hysteroscopes, the system can provide a visual or other feedback to guide hysteroscope 100 tip orientation to avoid or reduce perforation, pain, scraping, etc. This is especially applicable for cervixes with wavy lumens of varying sizes. One or more distances from anatomical walls may be mapped by the system to guide hysteroscope 100 tip orientation.

In some embodiments, as shown in FIG. 7, the steering of hysteroscope 100 may be performed using an optional robotic navigation system 710. For example, the steering or navigation of hysteroscope 100 may be assisted or performed by a robotic navigation system 710. Such a system may include one or more actuators, drive mechanisms, or robotic joints that impart controlled motion to hysteroscope 100 along one or more degrees of freedom. The robotic navigation system 710 may receive commands from a user interface, from automated control logic, or from a combination thereof, and may adjust the orientation or position of hysteroscope 100 accordingly. In some embodiments, the robotic navigation system 710 may operate in a semi-autonomous or fully autonomous mode in which the system adjusts the trajectory of hysteroscope 100 based on anatomical information extracted from the hysteroscopic images.

In some embodiments, hysteroscope 100 may also be flexible or comprise one or more steerable regions, as shown in FIG. 7 as optional steering / articulation Module 720. For example, hysteroscope 100 may include one or more flexible or steerable regions that permit deflection, bending, or articulation of the distal portion of the hysteroscope 100. The flexible or steerable regions may be manipulated manually, mechanically, or electromechanically to navigate through the cervical canal and/or the uterine cavity. In some embodiments, steering may be performed using pull wires, embedded actuators, or other suitable mechanisms for controlled bending of an elongate medical device. Such embodiments permit fine-tuned navigation around anatomical curves or toward anatomical landmarks such as the cornua.

In any of these embodiments, navigation or steering may be guided using visual feedback from hysteroscopic images, such as real time views of the cervical canal, uterine cavity, and/or cornual regions, for example as provided by guidance module 740. The system may display alignment indicators, trajectory markers, or other visual cues that assist the user or the robotic system in steering hysteroscope 100 toward a desired location. Additionally, or alternatively, the system may use other feedback sources (e.g., sensor(s) 760), such as positional sensors, orientation sensors, or data derived from prior anatomical mapping, to adjust or refine the steering of hysteroscope 100.

Any suitable generated information (e.g., size measurements, shape measurements, contours, navigation guides, etc.) in embodiments described herein may be conveyed to the user as a graphical overlay on a display device, for example using optional guidance module 740. In some embodiments, one or more insertion tract (external os, cervical canal, uterine cavity) parameters are measured or determined, for example using insertion tract measurement module 770. This may be used for various subsequent applications including, but not limited to: guiding the placement or insertion of devices such as intrauterine (e.g., IUDs, GEA devices) or intrafallopian tube devices. Examples of insertion tract parameters include, but are not limited to one or more of: lengths, distances, angles, orientations, anatomical landmarks, curves or bends, angles, notches, weak regions, regions at risk of perforation, etc. In some embodiments, the following insertion tract parameters are measured or determined: orientation of the cervical canal relative to the vagina, length of the cervical canal, orientation of the uterine cavity relative to the cervical canal, length of the uterine cavity, and various curves or bends of the insertion tract in the mid-sagittal plane. These parameters are then used to guide the placement of subsequent devices e.g., by steering one or more devices, adjusting the length or other dimensions of one or more devices, etc. One or more such parameters may be conveyed to a user through a graphical user interface, numeric displays, or other means.

In some embodiments, insertion tract parameters, such as those corresponding to the external os, cervical canal, or uterine cavity, may be determined by processing hysteroscopic image or video data obtained during advancement of the hysteroscope through the insertion tract. The system may identify anatomical boundaries or features visible within the hysteroscope 100 FOV and may use these features to derive geometric relationships, distances, orientations, or other quantitative characteristics of the insertion pathway. The measurement process may be carried out by insertion tract measurement module 770.

For example, as the hysteroscope 100 is advanced, the system 700 may continuously analyze the shape and position of the visible cervical canal walls to estimate the orientation of the canal relative to the vaginal axis. The system 700 may track changes in the appearance of anatomical structures across successive frames and may associate those changes with relative movement of the hysteroscope 100. Using this information, the system 700 may compute the length of the cervical canal by determining the distance traveled between identification of the external os and the internal os.

Similarly, upon entry into the uterine cavity, the system 700 may identify fundal regions, cornual openings, cavity contours, and/or other recognizable anatomical landmarks. By analyzing the spatial relationships among these landmarks across multiple video frames, the system 700 may derive orientations or angular transitions between segments of the insertion tract, including transitions between the cervical canal and the uterine cavity. The system 700 may also determine curves or bends in the tract by evaluating how the cavity profile or cervical canal axis changes along the mid-sagittal plane.

In some embodiments, the system 700 may apply one or more geometric, image processing, or feature extraction operations (e.g., using feature extraction module 754) to detect boundaries or reference points used in these measurements. Such operations may include identifying edges or contours of the tract, determining the centerline of the cervical canal, estimating curvature along that centerline, or detecting regions where the canal narrows, widens, changes orientation, or exhibits anatomical notches or irregularities. Regions identified as structurally thin or sharply angulated may be flagged as regions at elevated risk of perforation.

The parameters obtained, such as cervical canal length, uterine cavity length, canal-to-cavity orientation, curvature of the insertion tract, and angles at notable anatomical transitions, may be used to guide placement of subsequent devices. For example, the system may steer a device along a recommended trajectory, may adjust or recommend adjustments to the length or configuration of devices to be inserted, or may present indicators showing expected path curvature. One or more such parameters may be displayed to the user through a graphical interface, overlaid visual markers, numeric indicators, or other suitable presentation methods.

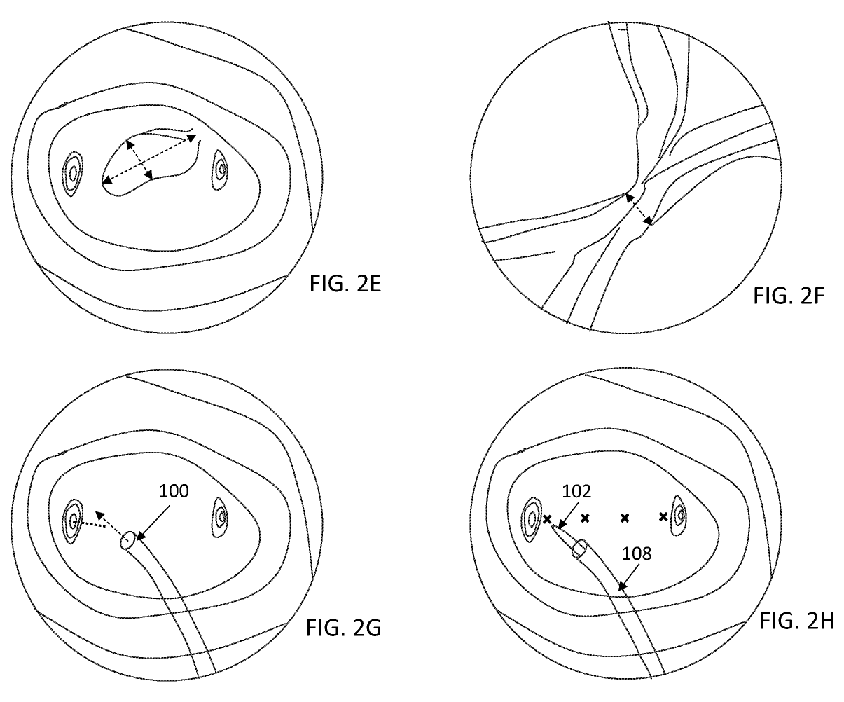

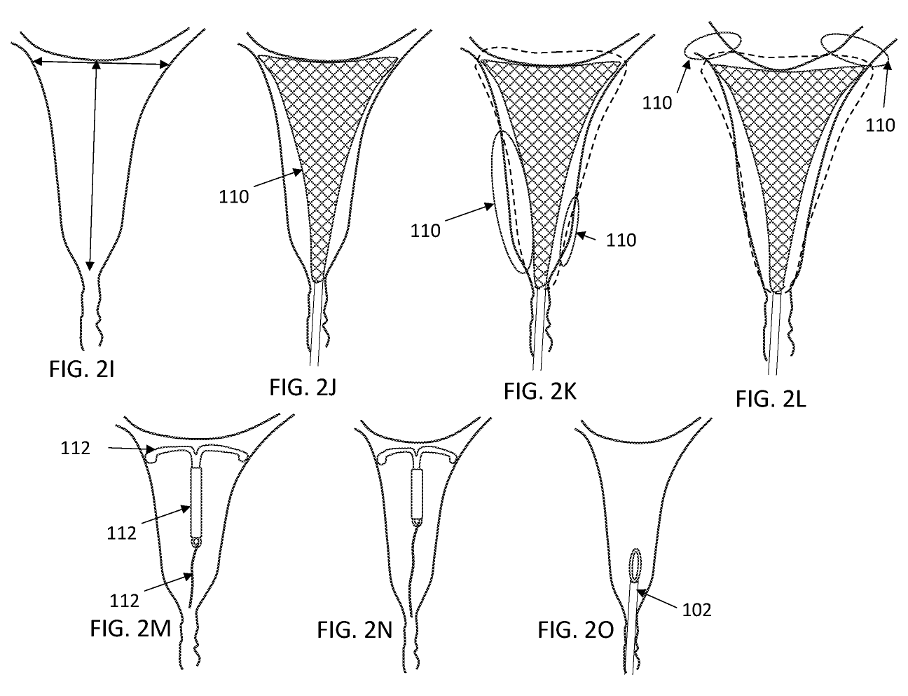

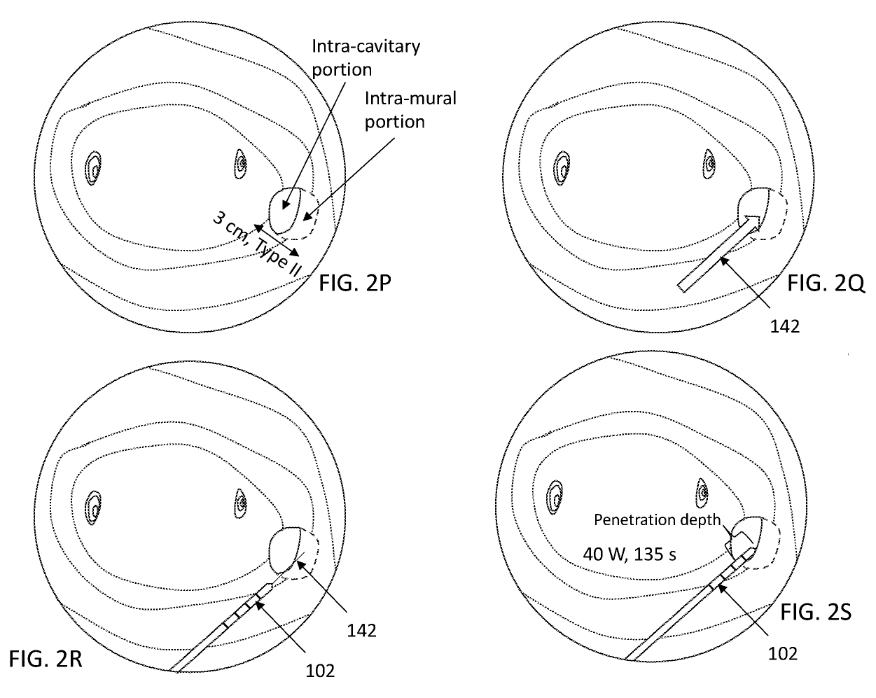

The information gathered by the system 700 may be processed to generate one or more outputs; examples of which include, but are not limited to: anatomical maps, anatomical dimensions, 2-D representations of three-dimensional regions, reconstructed views of one or more cavities or other regions, etc. Several such examples are shown in FIGS. 2A to 2O. To standardize imaging across multiple types of uterine cavities, as shown in FIG. 7, one or more visualization markers 780 (e.g., visual markers, color markers, size markers, etc.) may be introduced into the anatomy and visualized by hysteroscope 100 (e.g.,, using one or more imaging sensors 704 or sensor(s) 760). One or more outputs of the various embodiments of this disclosure may be used to guide, select or tailor one or more subsequent devices (e.g., an intrauterine device or IUD) and/or procedures (e.g., endometrial ablation, intrauterine access, etc.). FIG. 2A shows an embodiment of the system where the uterine cavity width has been calculated. In this embodiment, the uterine cavity has been calculated as the distance between the two cornua. Other methods of calculating the uterine cavity width may also be used. Uterine cavity width may be used as an input for subsequent procedures or calculations. For example, an IUD may be selected for implantation into the patient based on the uterine cavity width. In any of the embodiments described herein, the system 700 may be designed to allow the user to manually annotate one or more regions of the anatomical cavity. For example, the system 700 may be designed to receive user input, to allow, in some embodiments, manual annotation of one or more cornua, fibroids and other pathology that is seen in endoscopic images, etc.

FIG. 2B shows an embodiment of the system 700 wherein the deployment of an endometrial ablation device (i.e., other device 790) may be predicted by the system 700. In the embodiment shown, the maximum width of the endometrial ablation device has been calculated by the system 700 (e.g., using insertion tract measurement module 770). This information may be used to guide the deployment of such endometrial ablation devices. In some method embodiments, the actual width of a deployed endometrial ablation device is compared to the width calculated in FIG. 2B. If the actual width is lower than a predicted width, the endometrial ablation device may be redeployed, such that it covers a maximum surface, or substantially maximum surface, of the uterine cavity. If the actual width is greater than a predicted width, the cavity may be evaluated for perforation. Further, the length of the uterine cavity to be ablated may also be calculated in the embodiment shown in FIG. 2B. In some such embodiments, this length may be calculated based on the distance from the mid-fundus to the internal os. In some embodiments, an endometrial ablation device may be deployed (fully or partly) in the cavity such that the distal end of the endometrial ablation device is located around the mid region or at a distal region of the cavity without touching the fundus. Thereafter, one or more tests (e.g., perforation detection tests, deployment tests, cavity contour measurement steps, etc.) may be performed. In such embodiments, the insertion depth of the endometrial ablation device may be determined using the cavity size and shape data obtained from any of the methods described herein.

FIG. 2C shows an embodiment of the system, wherein the location of an IUD (i.e., other device 790) has been predicted and/or calculated by the system. Variations of the location of the IUD may be calculated and displayed by the system. Various types of IUDs may be similarly modeled to allow selection of an optimal IUD for the patient. One or more embodiments of the present disclosure may be used to predict one or more side effects of the IUD, predict the risk of inadvertent expulsion of the IUD, calculate one or more parameters to be used while customizing a customizable IUD, determining one or more therapy (example, drug dose) parameters of the IUD, etc. As shown in FIG. 2C, the IUD is shown to be fitting well inside the uterine cavity.

I n some embodiments, a system 700 may optionally execute one or more methods of device customization module 792. Fr example, device customization module 792 may determine a predicted position of an IUD (i.e., other device 790) within a uterine cavity based at least in part on anatomical information previously acquired from hysteroscopic images and/or videos, from reconstructed cavity profiles, and/or from insertion tract parameters as described herein. The system may store or access a device model representing one or more geometric, mechanical, and/or deployment characteristics of the IUD. The device model may include, without limitation, overall length, arm span, stem geometry, nominal fundal seating reference, and one or more allowed deformations or articulations during or after deployment. The system may register the device model to a uterine cavity representation by aligning one or more reference points (e.g., fundal region, mid-sagittal axis, cornual regions) and may compute a predicted IUD position and orientation upon deployment.

In some embodiments, device customization module 792 may generate positional variations of the device model to reflect potential intra-procedural and post-procedural changes. By way of non-limiting example, the system may apply translations, rotations, flexural deformations, and/or cavity distension adjustments to the device model and may evaluate each variation relative to the uterine cavity representation. The variations may include differences in fundal seating depth, lateral arm tilt, stem angulation relative to the cervical canal, and/or asymmetry relative to the cavity midline. The system may display the predicted position and one or more variations to a user, for example as graphical overlays on a reconstructed view, as a wireframe within a 3D map, and/or as numerical indicators of distances, angles, and clearances.

In some embodiments, device customization module 792 may evaluate a fit metric or goodness-of-fit score between the device model and the uterine cavity representation. The fit metric may be based on one or more of: distance between device extremities and the cavity boundary at defined locations (e.g.,, fundal roof, lateral walls at the level of the mid-fundus); angular alignment between a device axis and a cavity axis; estimated tissue-contact distribution along the device arms and stem; and/or expected symmetry relative to a cavity midline. The fit metric may be used to select a device from among a set of candidate IUD models and/or to parameterize a customizable IUD.

In some embodiments, device customization module 792 may compute one or more risk indicators associated with the predicted position of IUD. Without limitation, the risk indicators may include indicators of suboptimal fundal seating, lateral or cornual embedding, malposition relative to the cervical canal, inadvertent expulsion, perforation risk, and/or side-effect likelihood (e.g., pain, cramping, bleeding).

The device customization module 792 may determine a fundal gap distance between a superior portion of the device model and a fundal reference surface. A fundal gap exceeding a threshold value and/or a device tilt exceeding a threshold angle relative to a cavity axis may be flagged as a fundal malposition condition. Thresholds may be fixed, user-selectable, or adaptively determined based on measured cavity size and shape. The system may present a warning or may recommend repositioning or selection of an alternative device size.

In some embodiments, the device customization module 792 may compute lateral clearances between each device arm and a lateral wall at the fundal level and/or between each arm and an identified cornual recess. Regions of negative clearance (predicted interpenetration) or regions of contact exceeding a contact-pressure proxy (e.g., high curvature of the cavity surface relative to arm geometry) may be flagged as a potential embedding risk. The system may recommend a reduced arm span, alternative device geometry, or a modified deployment approach.

In some embodiments, the device customization module 792 may compute an axis alignment score between a device longitudinal axis and a cavity-to-cervical canal axis. A low alignment score, a low fundal seating score, and/or a predicted inferior displacement under nominal physiologic perturbations (e.g., modeled cyclical cavity contractions or pressure changes) may be aggregated into an expulsion risk index. In some embodiments, the expulsion risk index is evaluated across the aforementioned positional variations to identify worst-case scenarios.

In some embodiments, the device customization module 792 may identify regions of concern within the uterine cavity based on anatomical features previously detected (e.g.,, sharply curved segments, thin regions, notches, or regions designated as at risk of perforation). The system may project the device model and one or more post-deployment arm trajectories into those regions and compute a proximity or approach angle metric. If the approach angle and proximity exceed defined criteria, the system may issue a perforation-risk warning, may suggest a different insertion trajectory, or may constrain steerable deployment to avoid the flagged region.In some embodiments, the device customization module 792 may calculate contact distribution features between the device model and the cavity surface (e.g.,, magnitude and spatial extent of predicted contact, asymmetry of contact between left and right arms, or contact near sensitive anatomical landmarks). The system may associate such features with a side-effect likelihood indicator for outcomes such as pain, cramping, or bleeding. The indicator may be used to recommend an alternative device geometry and/or a modified fundal seating depth. In certain embodiments, the device customization module 792 may store a library of device models representing different IUD geometries, sizes, and configurations. The system may evaluate the fit metric and risk indicators for each candidate device and may present a ranked selection to the user. For a customizable IUD, the system may compute recommended parameters (e.g., arm span, stem length, drug-eluting surface area or dose rate, deployment angle) based on the cavity dimensions, predicted fundal seating, and the aforementioned risk indicators. The computed parameters may be used to configure the customizable device prior to deployment and/or to adjust a steerable or actuated device intra-procedurally.

The device customization module 792 may present one or more of the predicted device position, positional variations, fit metrics, and risk indicators via a user interface, for example of guidance module 740. Non-limiting examples include color-coded overlays on a reconstructed 2D or 3D view, numeric readouts of distances and angles, bar or gauge indicators for risk indices, and/or textual prompts recommending device selection or deployment adjustments. In some embodiments, the guidance output may be used to automatically or semi-autonomously adjust a device trajectory, steer a deployment tool, and/or impose virtual constraints that limit device motion within regions of concern.

FIG. 2D shows another embodiment of the system, wherein the location of an IUD has been predicted and/or calculated by the system. In this embodiment, the uterine cavity width is smaller than the width of the IUD. In such situations, the system may provide feedback (e.g., using guidance module 740) that the IUD is unsuitable for the patient. One or more embodiments of the present disclosure may be used to determine and/or provide feedback about the suitability of one or more therapeutic interventions of the uterus.

FIG. 2E shows an embodiment of the system for measuring intrauterine pathology. In this embodiment, a polyp has been detected by the system, and the dimensions of the polyp have been measured by the system. Any of the measurements described here may be performed by imaging one or more anatomical regions from multiple locations by endoscope 100. One or more computer vision or artificial intelligence techniques (e.g., using computer vision / AI module 794 and/or 2D or 3D model generation module 750) may be used to create a 3D profile of such lesions, and the dimensions of such lesions may be displayed or otherwise communicated using system 700. In one embodiment, 3Ds of a polyp or fibroid are calculated and/or communicated to the user.

In some embodiments, as shown in FIG. 7, system 700 may employ one or more computer-vision or artificial intelligence (AI) techniques (e.g., computer vision / AI module 794) to detect intrauterine pathology and determine one or more dimensions of such pathology. For example, the system 700 may analyze hysteroscopic image or video frames to identify visual features associated with a polyp, fibroid, or other lesion. The system 700 may apply any suitable image processing or feature extraction operation (e.g., using feature extraction module 754), including but not limited to edge detection, contour extraction, region segmentation, texture-based classification, or landmark detection, to delineate the boundaries of the lesion from surrounding tissues.

In some embodiments, the system 700 may further generate a 3D profile of the lesion using information derived from imaging the anatomical region from multiple locations during movement of endoscope 100. For example, the system 700 may track the appearance of the lesion across frames acquired from differing viewpoints and may estimate relative depth, curvature, or surface geometry based on changes in perspective. The system 700 may use any suitable 3D reconstruction technique, including stereo-correlation, multi-view integration, depth-from-motion analysis, or AI-based depth estimation models, to create a volumetric or surface representation of the lesion. The resulting 3D representation may be used to calculate one or more dimensions of the lesion, such as height, width, depth, surface area, or volume.

In some embodiments, computer vision / AI module 794 may use one or more AI models, such as machine-learning or deep-learning models trained on annotated hysteroscopic data. Computer vision / AI module 794 may be used to detect lesion boundaries, classify lesion types, or refine 3D estimates. Such models may assist in identifying subtle features or in improving segmentation accuracy in complex visual environments. The calculated lesion dimensions or 3D representations may be displayed to the user, incorporated into measurement reports, or used to guide subsequent interventions.

FIG. 2F shows an embodiment of the system where in one or more adhesions have been analyzed by the system. One or more size and/or location parameters of such adhesions may be calculated and/or communicated to the user. This information may in turn be used to plan subsequent procedures, such as adhesion lysis.

In some embodiments, system 700 may analyze hysteroscopic image or video data obtained by endoscope 100 to detect one or more intrauterine adhesions. The system 700 may apply any suitable computer vision or image processing techniques (e.g., using computer vision / AI module 794) to identify visual features associated with adhesions, including variations in surface continuity, tissue texture, reflectance, color characteristics, or contour irregularities. For example, the system 700 may perform boundary detection, region segmentation, contour extraction, or texture-based feature analysis to distinguish adhesion tissue from adjacent endometrial surfaces.

In some embodiments, the system 700 may generate a 3D representation of an adhesion by analyzing images of the adhesion captured from multiple viewpoints as the endoscope is moved within the cavity. The system 700 may estimate relative depth, thickness, or surface geometry of the adhesion using multi-view correlation, depth-from-motion analysis, or other suitable reconstruction techniques, including AI-based depth estimation or segmentation models trained to recognize adhesion morphology. From the reconstructed geometry, the system 700 may calculate one or more parameters of the adhesion, including length, width, height, thickness, surface area, volume, location within the uterine cavity, proximity to anatomical landmarks, and extent of cavity occlusion.

In some embodiments, the system 700, using computer vision / AI module 794, may further classify adhesions according to severity, density, or degree of cavity involvement by evaluating the spatial characteristics of the adhesion relative to the surrounding cavity. These calculated characteristics may be used to plan subsequent procedures, such as adhesion lysis, by identifying optimal access points, recommended instrument trajectories, or regions where the adhesion may be more safely or effectively separated. The measured adhesion parameters may be displayed or otherwise communicated to the user, for example through graphical overlays, numerical indicators, or 3D models shown on a display, for example of guidance module 740 associated with system 700.

FIGS. 2G shows a step of a method of accessing the cornua or the fallopian tube lumen using an embodiment of the present disclosure. In FIG. 2G, the system provides a visual guide for orienting an access device into a cornu or fallopian tube lumen.

FIG. 2H shows an embodiment of the system for directing injection of local anesthesia or other medications. The system may mark one or more sites in one or more images. The sites may be determined by one or more of: cornual anatomy, fundal anatomy, etc. Sites for injecting local anesthesia or other medications may be determined using computer vision techniques (e.g., using computer Vision / AI module 794). The injection into such sites may be guided by computer vision-based identification or analysis.

FIGS. 2I through 2L show various steps of method embodiments of predicting the performance of endometrial ablation inside a uterine cavity. FIG. 2I shows mapping one or more cavity dimensions. FIGS. 2J and 2K show determining the deployment of an endometrial ablation device and calculating regions expected to remain unablated in the lower uterine region. FIG. 2L shows determining the deployment of an endometrial ablation device and calculating regions expected to remain unablated in the cornual regions.

FIGS. 2M and 2N show various method embodiments of selecting an IUD for the uterine cavity based on cavity size and other anatomical characteristics or parameters. Such selection may be done for reducing side effects, reducing risk of expulsion, increasing patient satisfaction, etc., as described elsewhere herein.

FIG. 2O shows a device being used to treat or a diagnose a portion of the uterine cavity wherein the insertion, deployment, movements, or actions of the device are based on information obtained prior to the insertion of this device. A curette is used as an example in FIG. 2O. Other examples of devices include, but are not limited to, suction devices, cutting devices, biopsy devices, ablating devices, etc. In some embodiments, suction devices or other soft tissue removal devices may be used to remove floating debris or other material that is obstructing the view of one or more regions of the endometrium.

FIGS. 2P – 2S show various embodiments of methods and devices to treat pathology located within a wall of an anatomical cavity. In FIGS. 2P – 2S, fibroids located within uterine walls are used as an example to illustrate some details of the device and method embodiments. Other pathologies that can be treated with similar devices and methods include, but are not limited to malignancy, benign tumors, adenomyosis, and abnormal vasculature.

In the step of FIG. 2P, a hysteroscope is used to image the uterine cavity. A fibroid located within the walls of the uterus is visualized using the endoscope 100. In the example shown, the fibroid has an intra-cavitary portion and an intra-mural portion. One or more processing steps described herein using one or more hardware components (e.g., feature extraction module 754 and/or computer vision/AI module 794 of system 700 shown in FIG. 7),) described herein may be used for one or more of:

Determining one or more fibroid parameters including, but not limited to: size, shape, location, type, hardness, etc.

Displaying intramural portion of the fibroid on a display

Determining type (e.g., resection, morcellation, ablation, injecting, liquefaction, etc.) of therapy to be performed

Determining entry point of a device into the anatomy or fibroid to target the centroid of the fibroid.

Determining orientation of a working device 102 to reach the centroid of the fibroid, etc.

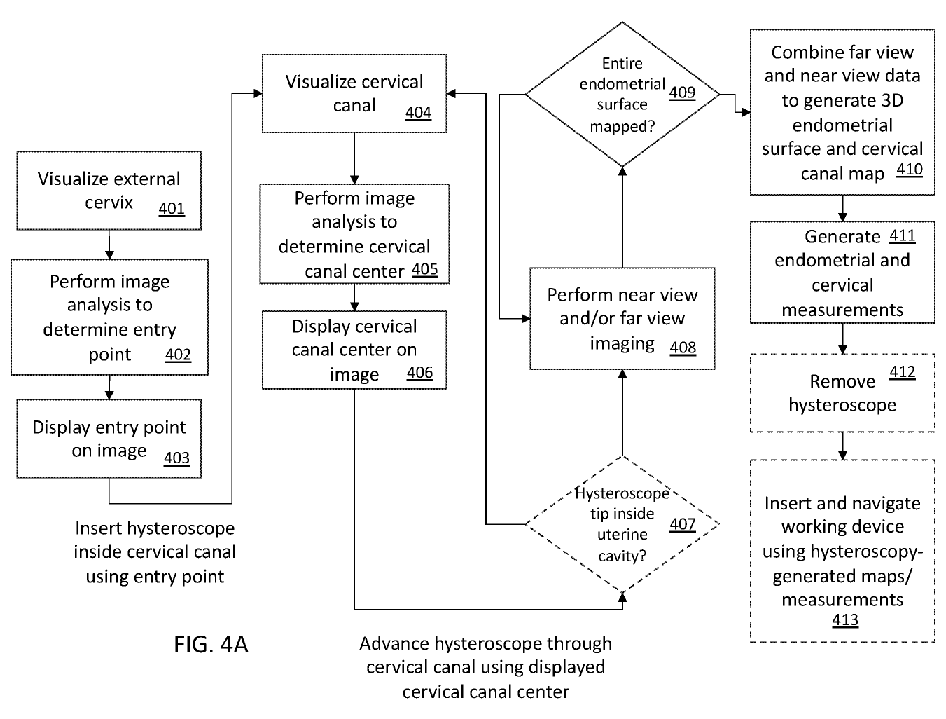

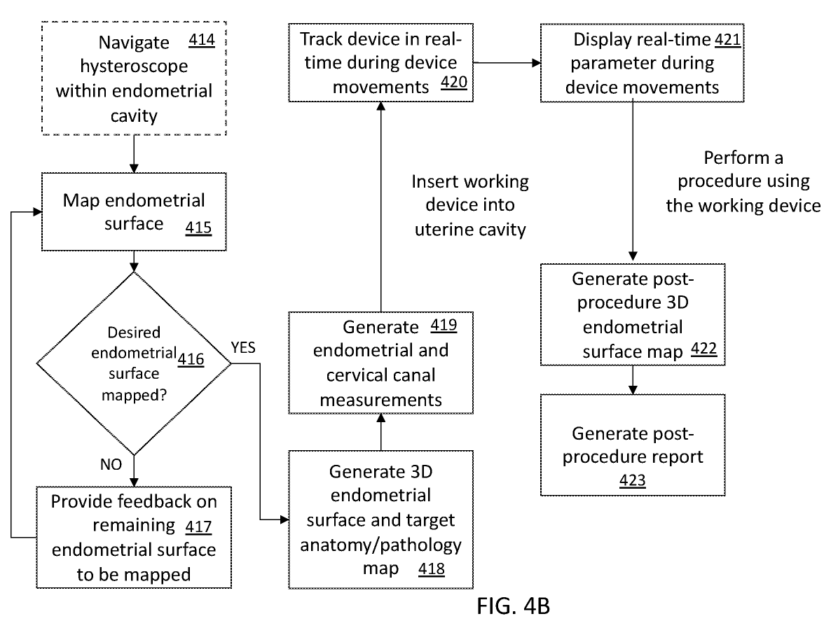

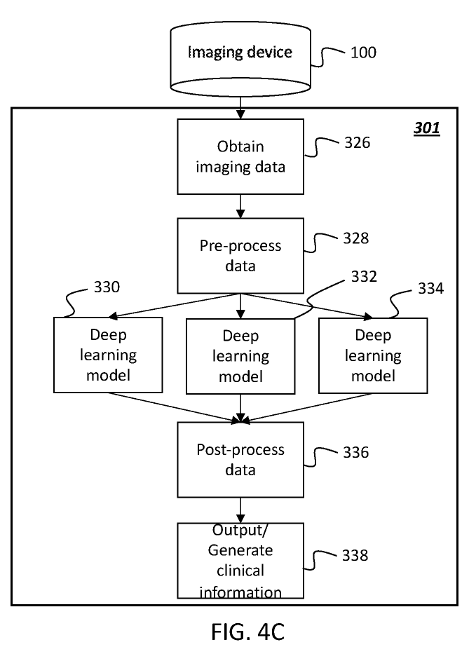

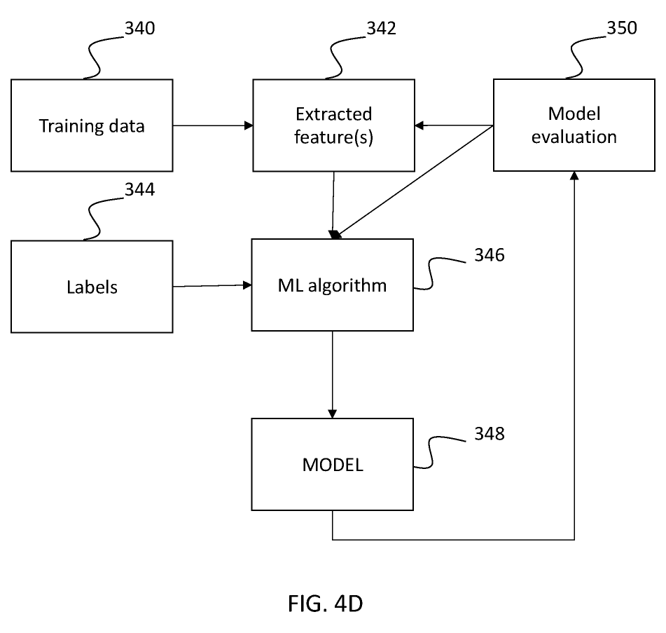

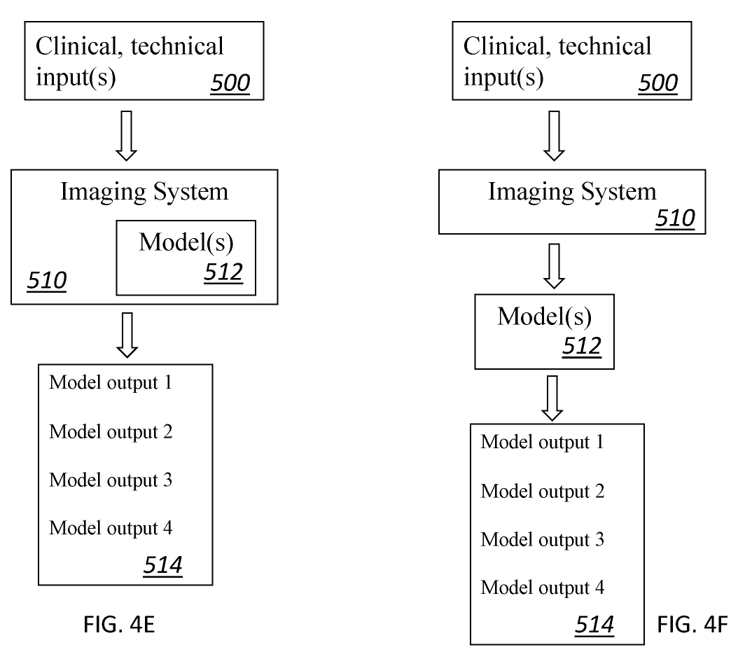

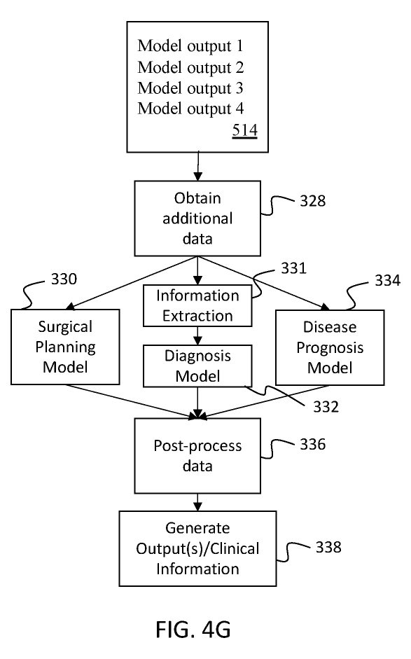

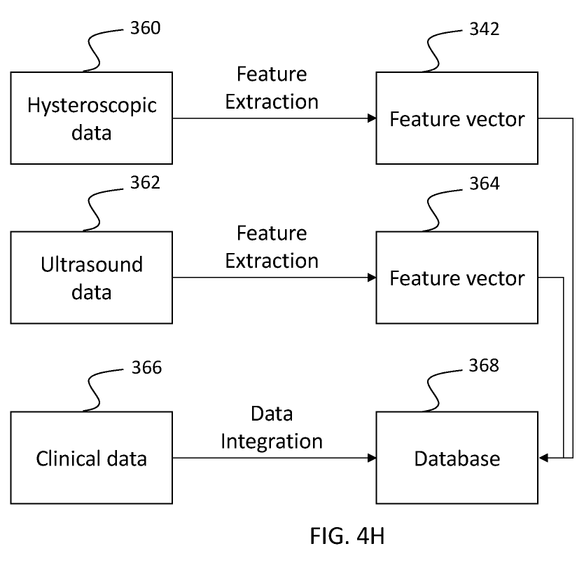

In any of the methods disclosed herein, additional imaging data (e.g., ultrasound or MRI data) may be combined with hysteroscopic imaging data to generate a 3D representation of the uterine walls and/or determine and/or carry out one or more processing steps (examples described above), etc. One embodiment of combining imaging data is described in FIG. 4H. Embodiments wherein multiple imaging data is combined are especially suitable to treat deeper pathology (e.g., Type III-VI fibroids) that is not visible in hysteroscopic imaging data. Hysteroscopic imaging may be used to guide a working device 102 (e.g., shown in FIG. 7) in real-time along a path that would pass through such a fibroid.

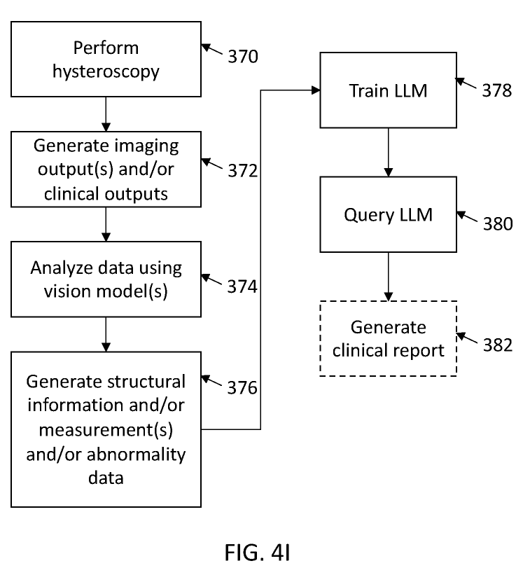

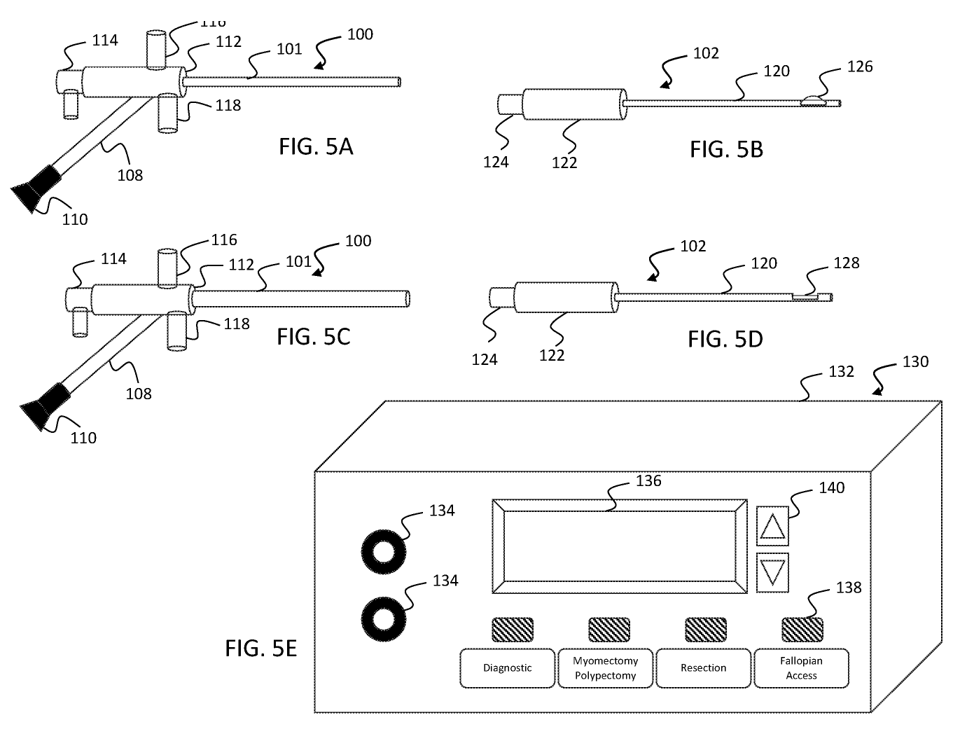

In FIG. 2Q, an access path is calculated by the system to enter the fibroid. The access path may be displayed on a display as a guide icon 142 (e.g., an arrow, a line, etc.). In FIG. 2R, a working device 102 (e.g., an ablation device) is introduced into the uterine cavity using the guide icon 142. The guide icon 142 may guide the user in one or more ways including, but not limited to: guiding the orientation of working device 102 and/or showing the point of penetration into the cavity wall, etc. Guide icon 142 may be updated in real time depending on the position of pathology, position and/or orientation of working device 102, etc. In FIG. 2S, the working device 102 is used to penetrate the fibroid using guide icon 142. The calculated, desired penetration depth of working device 102 may be displayed on a display. The penetration depth of working device 102 may be determined by the user and/or the system using one or more external markers (e.g., visual markers) on working device 102, computer vision techniques, etc. The user may advance working device 102 into the cavity wall until the desired penetration depth of working device 102 is achieved. Thereafter, one or more therapies (e.g., ablation) may be performed using working device 102. Working device 102 may be any of the devices disclosed herein including, but not limited to: injecting devices, morcellating devices, liquifying devices, ablating devices, etc. In some embodiments, working device 102 may penetrate an endometrial region before entering a fibroid. Treatment settings, working parameters, etc. of working device 102 may be determined using any of the method and device embodiments disclosed herein. Such automated calculate of one or more working parameters may be performed in any suitable embodiments described herein. These working parameters may be conveyed to a user or may be automatically applied (e.g., in the embodiment shown in FIG. 5E) during the use of working device 102.

Methods such as those shown in FIGS. 2P – 2S may use various techniques to estimate the position and size of a mass (e.g., a fibroid) that is under the wall surface, but whose presence changes the wall surface. Various computer vision techniques may be used for this estimation. Computer vision techniques (e.g., using computer vision / AI module 794) based on subsurface scattering may be used to infer differences in structures underlying the cavity wall and estimate the shape or size of such structures. Shape-from-shading techniques may be used that analyze variations in brightness within a single image to infer the 3D shape of a surface. If a surface is facing a light source, it appears brighter. If the surface is angled away, it appears darker. By analyzing these intensity differences, computer vision algorithms may estimate the orientation of the surface at each point. Bulges or deformations created by objects underneath a cavity surface may then be estimated and used to detect the presence of sub-surface objects such as fibroids and estimate their size and location. In some embodiments, photometric stereo techniques are used to detect the presence of sub-surface objects such as fibroids and estimate their size and location. In some embodiments, deep learning models (e.g., using computer vision / AI module 794) that are trained to map surface images directly to estimates of shape, depth, or hidden structure may be used. In such methods, neural networks may be trained to learn patterns from large datasets. For example, they may be trained to learn how lighting, material properties, color and/or texture variations and subsurface effects influence hysteroscopic images. In some embodiments, the dimensions and center of mass (centroid) of a subsurface object may be estimated using computer vision algorithms that integrate geometric models, system calibration data, and pixel-level processing.

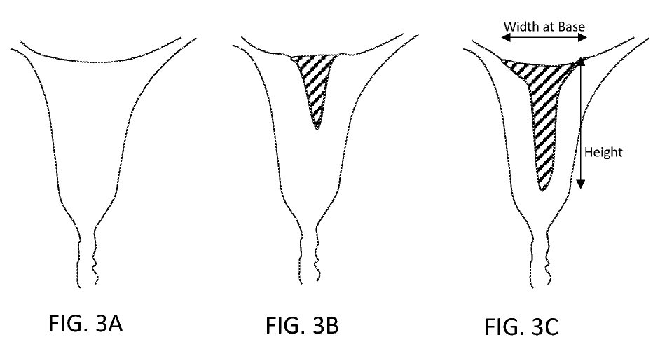

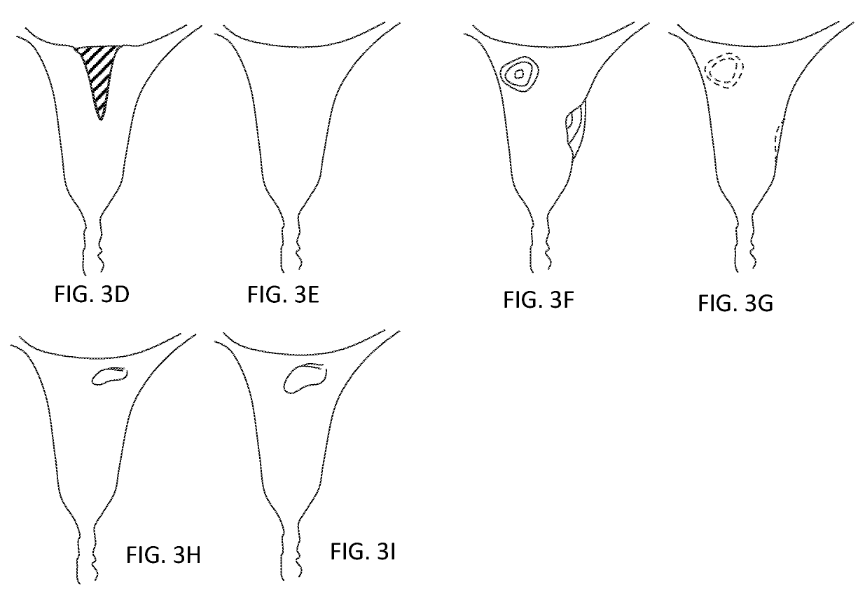

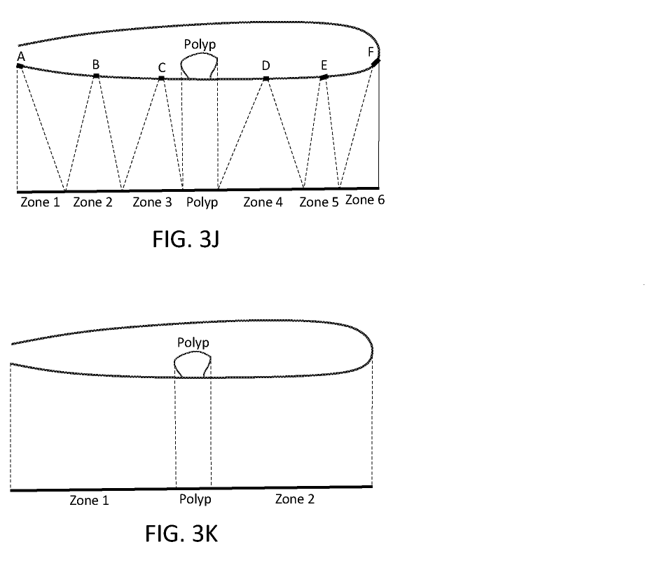

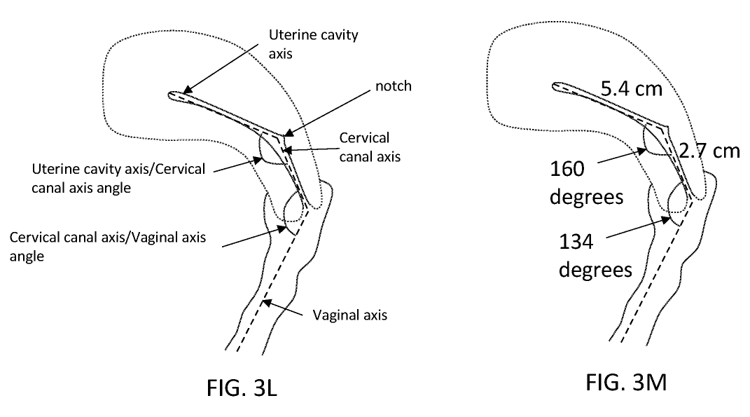

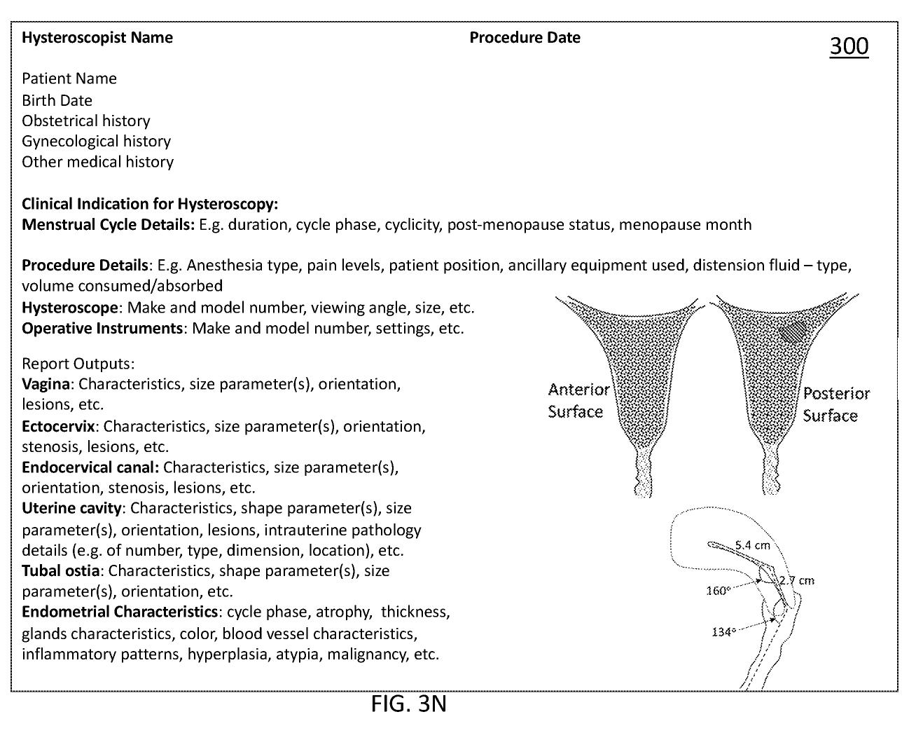

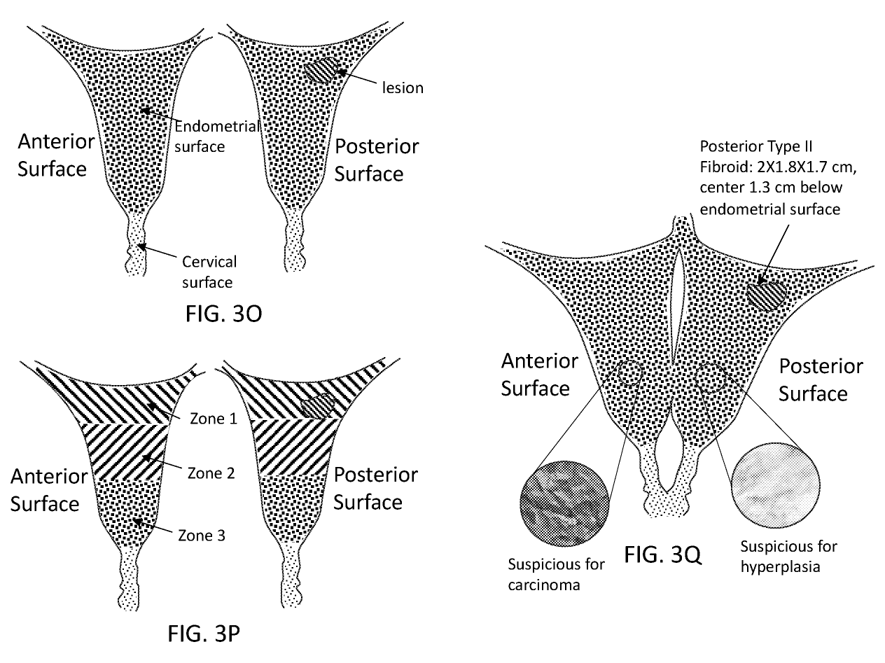

FIGS. 3A – 3C show embodiments of visual outputs of some embodiments of the present disclosure wherein one or more pathologies have been marked. In FIG. 3A, there is no pathology seen and the cavity outline is marked. FIGS. 3B shows a septum. Multiple size and shape parameters of such pathologies may be calculated and documented. FIGS. 3C shows an almost complete septum. Multiple size and shape parameters of septums and other pathologies may be calculated and documented. FIG. 3C shows an example of calculating and documenting size (e.g., width at base, height) and/or shape parameters of a septum.

Embodiments may be used for documenting the change in uterine anatomy and/or pathology overt time. FIGS. 3D and 3E show images of the uterine cavity obtained by some embodiments before and after septoplasty. FIGS. 3F and 3G show images of the uterine cavity obtained by some embodiments before and after myomectomies.

Embodiments may be used for tracking the natural and/or pathological changes in the uterus over time. FIGS. 3H and 3I show images of the uterine cavity at two time points showing the growth of a polyp.

In some method embodiments, using feature extraction module 754 and/or computer vision / AI module 794 of FIG. 7, hysteroscopic data (e.g., 3D reconstructions of the uterine cavity, pathological data, etc.) may be compared across two or more timepoints. This may be performed using methods including, but not limited to: feature-based matching, object tracking, and/or object re-identification/correspondence. Feature matching algorithms identify distinctive features in each image and then try to match them across time points. Examples of features include, but are not limited to: corners (e.g., cornua), edges, textured regions, projecting regions, etc. Such algorithms may be one or more of: Scale-Invariant Feature Transform (SIFT), Speeded Up Robust Features (SURF), Oriented FAST and Rotated BRIEF (ORB), and Binary Robust Independent Elementary Feature (BRIEF). Features may be identified using deep neural networks that may capture complex patterns like shape, texture, and object context.

Visual data of the cavity surface may be converted to a 2D format for display, printing on a report, etc. In the embodiment shown in FIG. 3J, regions of the uterine cavity are magnified and mapped to zones of the output image. For example, region A has been magnified and displayed on Zone 1 of the output image. The uterine cavity dimensions and shape may be displayed without scaling. The pathology (e.g., polyp) may also be mapped without scaling to allow an estimation of the true size and/or shape of the pathology and the uterine cavity. FIG. 3K shows another embodiment wherein the curved uterine surface has been projected onto a 2D surface. In some embodiments, the entire uterine cavity surface may be imaged and converted to a 2D format. In method embodiments, the user may be prompted and/or guided if a particular region of the uterine cavity has not been adequately imaged.

FIGS. 3L and 3M show embodiments of an output image of the present disclosure showing various orientations, sizes, distances, angles, etc. Such a view of the anatomy may be obtained by combining the data and/or measurements made during inserting and/or withdrawing and/or navigating hysteroscope 100. For example, sequential view of the vagina, external os, cervical canal, and the uterine cavity (e.g., those from the method shown in FIGS. 1A – 1G) may be combined to generate one or more measurements or data shown in FIG. 3L. Any of the output images or other outputs disclosed herein may contain mapping data of key anatomical landmarks (e.g., vagina, cervical external os, cervical canal, internal os, endometrial cavity, etc.) to subsequently guide (e.g.,, using guidance module 740) and facilitate smoother and safer insertions of devices (e.g., IUDs) inserted into the uterine cavity or other uterine regions. This is especially advantageous for nulliparous patients, who often experience higher levels of discomfort and pain during intrauterine procedures.

In some embodiments, the uterocervical angle (UCA) and/or cervical length may be measured using one or more embodiments described herein. UCA is the angle between the anterior uterine segment and the cervical canal. These and other measurements may be used to predict one or more outcomes including, but not limited to: risk of preterm birth, embryo transfer success rate, severity of primary dysmenorrhea, risk of IUD expulsion, risk of IUD malposition, etc.

In some embodiments, the system 700 may calculate the uterocervical angle (UCA) and/or cervical length by analyzing hysteroscopic image or video data acquired as endoscope 100 is advanced through the cervical canal and into the uterine cavity. In some embodiments, this analysis is performed using one or more modules such as feature extraction module 754 and/or computer vision/AI module 794, and/or 2D or 3D Model Generation Module 750 of system 700 shown in FIG. 7. The system may identify anatomical boundaries corresponding to the cervical canal walls, the internal os, and the anterior uterine segment using any suitable geometric, image processing, or feature extraction operations. For example, the system may extract canal centerlines, detect curvature transitions, or identify fundal and cervical landmarks based on contour changes or texture differences visible within the endoscopic field of view.

To calculate cervical length, the system may track the relative position of the endoscope as it progresses from the external os to the internal os and may compute the distance between these anatomical landmarks using frame-to-frame motion data, geometric calibration of the endoscope tip, or depth-from-motion estimation. In other embodiments, the system may estimate cervical length by generating a reconstructed mid-sagittal profile of the cervical canal and calculating the length of the centerline of the reconstructed canal path.

To calculate the UCA, the system may determine an orientation vector representing the anterior uterine segment and a second orientation vector representing the cervical canal axis. The anterior uterine segment orientation may be identified using cavity contours or fundal geometry observed after the endoscope enters the uterine cavity. The cervical canal axis may be determined by fitting a line, spline, or other geometric representation to the centerline of the canal or detected canal contours. The UCA may then be computed as the angle between these vectors using standard geometric or trigonometric operations. In some embodiments, the system may refine this calculation by smoothing centerline estimates, filtering noise, or incorporating additional anatomical reference points.

In some embodiments, the calculated UCA and cervical length may be used by the system to predict one or more clinical outcomes associated with uterine or cervical anatomy. Non-limiting examples include predicting the risk of preterm birth, predicting embryo-transfer success rates, evaluating the severity of primary dysmenorrhea, and predicting risk of IUD expulsion or malposition. The system may display such calculated parameters to the user via numerical readouts, graphical overlays, angular indicators, or other suitable presentation methods, and may optionally use these parameters to guide device selection, insertion trajectory, or procedural planning.

After any of the anatomical mapping or determination or documentation disclosed herein, one of more subsequent procedures may be performed. For example, intrauterine devices may be inserted using any of the data described herein. In one such example, an IUD inserting device (e.g., other device 790) is inserted into the uterine cavity following the anatomical mapping performed shown in FIG. 3M. Such an inserting device may be adjusted, for example, using the anatomical data shown. Such an inserting device may be curved or contain a steerable or deflectable region(s). The insertion of devices in subsequent procedures may be carried out after accounting for the various lengths, distances, curves, notches, regions where there is a risk or perforation or obstruction, etc. determined by one or more embodiments herein.

FIG. 3N shows an embodiment of a hysteroscopy report 300 produced by some embodiments of the present disclosure. The report 300 may comprise a data structure or visual representation of the uterine cavity. This may be used to detect changes with time. The changes may be analyzed to determine things like fertility, infertility, growth of lesions, endometrial quality change, menopausal status, etc. In one embodiment, such data structure or visual representation are designed such that the entire uterine cavity is visible in a single view. In some embodiments, one or more device or method embodiments are used to generate a digital high resolution image file or a file in other format. The file may contain any of the outputs described herein. In some embodiments, the file may contain an image that represents the entire uterine cavity. Such image files may be sufficiently detailed, for example, high resolution (e.g., > 300 or >600 DPI) or have a large file size, for example, greater than 1 MB, to capture detailed information about the uterine cavity. Such files may be used as inputs to train AI or other models or otherwise generate clinically useful information. One or more portions of the report (e.g., endometrial characteristics, sizes, distances, etc.) may be generated automatically.

The clinical terms in such generated reports may be in the form of SNOMED CT (Systematized Nomenclature of Medicine – Clinical Terms) to facilitate the use of the clinical information in the reports by EHRs, by AI models for diagnosis, by AI models for their training, by AI models for guiding one or more therapies (some examples of which are described herein), etc. The reports generated may comprise AI-analyzable structured elements such a texts and images. The reports generated may be used to train multi-modal AI models. The reports and other data generated from one or more embodiments herein may be used for training AI models such as foundation models, which in turn may be used for diagnosis and/or therapy.

FIGS. 3N – 3Q show examples of 2D visual/graphical representation of the uterine anatomy and/or pathology that may be generated by some device and method embodiments. Such 2D representation may have shape contours (examples shown in FIGS. 2I – 2O, 3N – 3Q, etc.) of the anatomical cavity, converted image contours (examples shown in FIGS. 3N – 3Q) of a 3D region of the anatomical cavity, outline of a section (examples of a mid-sagittal section shown in FIGS. 2I – 2O) through a plane of the anatomical cavity, etc. One or more visual effects such as shadowing may be added to solid pathology (e.g., fibroids, polyps, etc.) or cavity abnormalities (e.g., Müllerian anomalies) for easier understanding of the 3D structure of such elements in 2D representations. In some embodiments, a 3D reconstruction of the uterine cavity and cervical surfaces may be generated and stored and/or displayed. One or more size and/or shape parameters may be displayed in such representations. Endoscopic views of the cavity surface may be generated and displayed graphically. In some embodiments, various zones of the uterine cavity may be displayed and/or marked. In some embodiment, areas of suspected pathology (e.g., hyperplasia, cancer, polyps, fibroids, etc.) may be identified and/or marked in such representations. Various fibroid parameters such as type, size, shape, location below the cavity surface, etc. may be calculated and/or displayed.

FIGS. 4A to 4G show various method embodiments of the present disclosure. FIG. 4A shows a method embodiment of a hysteroscope procedure. The data generated from the hysteroscopy procedure may be used for subsequent procedures. Some examples of subsequent procedures are mentioned elsewhere herein. These include, but are not limited to, procedures (e.g., polypectomy, myomectomy, adhesion lysis, etc.) performed with robot assistance. In some embodiments, a subsequent hysteroscopy is performed using data generated from a prior hysteroscopy procedure. Far view imaging may be performed with the hysteroscope tip (e.g., using image sensor 704) or other imaging element (e.g., sensor(s) 760) positioned farther from the cavity wall to visualize a larger region of the cavity wall. Near view imaging may be performed with the hysteroscope tip or other imaging element positioned nearer to the cavity wall to visualize a smaller, magnified region of the cavity wall.

Hysteroscopic data (e.g., video streams, images, etc.) may be used for one or more of:

creating anatomical models of the uterine cavity or other anatomical regions,

constructing 3D or 2D maps of the uterine cavity or other anatomical regions,

measuring/calculating one or more of: size parameters, shape parameters, other detailed characteristics (e.g., dimensions, shape, contours, etc.), quantitative anatomical data, etc. of the uterine cavity or other anatomical regions,

generating detailed visual profiles of the uterine cavity or other anatomical regions, obtaining endometrial characteristics, and

obtaining cervical characteristics.

Anatomical models of the uterine cavity or other anatomical regions created by some embodiments may be combined with ultrasound data to create more detailed uterine models (e.g., using 2D or 3D model generation module 750). Such models may be used for any of the methods or purposes disclosed herein.

A variety of techniques may be used for these constructions and/or measurements, examples of which include, but are not limited to:

Structure from Motion (SfM) techniques. Such techniques reconstruct 3D structures from a series of 2D images taken from multiple viewpoints. When hysteroscope 100 is moved, multiple images may be captured from multiple viewpoints. Features in the anatomy are then tracked across frames. SfM algorithms may then be used to calculate depth and other parameters by analyzing how these features move relative to each other.

Photometric Stereo techniques. Such techniques estimate the surface normals of objects by observing that object under different lighting conditions (photometry).

Structured-light 3D scanning techniques. Such techniques capture the three-dimensional shape of an object by projecting light patterns (e.g., grids, stripes, etc.) onto the surface of the object. The deformation of such patterns may be recorded by hysteroscope 100 and processed using algorithms to obtain 3D data on the object.

Depth from Defocus (DfD) techniques. Such techniques capture images with different focal settings and analyze the degree of blur at each pixel to estimate depth.

SLAM (Simultaneous Localization and Mapping) techniques. SLAM techniques use a moving camera to map the environment while tracking (e.g., using sensors) the camera position in real-time.

Neural Radiance Fields (NeRF). Such techniques use deep learning to generate a 3D representation from a set of 2D images.

Single Image Depth Estimation Techniques. Such techniques (also known as Monocular Depth Estimation) may use AI models trained on large datasets to infer depth from single images. For example, such models may analyze texture, perspective cues, and object relationships to estimate relative depths.

In some embodiments, a 3D map or 2D map may be created and then processed to obtain one or more measurements disclosed herein.

Contour detection methods may be used to determine anatomical contours. Identified contours may be used for one or more of: classify the contours, identify the ROI(s) of anatomy/pathology, analyze one or more ROI(s) to determine pathology, etc.

FIG. 4A illustrates an embodiment of a hysteroscopic workflow in which system-generated imaging, analysis, and mapping steps guide both the acquisition of anatomical data and the preparation for subsequent procedures. The process begins by visualizing the external cervix (block 401) using endoscope 100, allowing the system 700 to analyze the viewed structures and determine an entry point of the cervix by identifying anatomical boundaries and contours (block 402). A graphical indicator representing the entry point may be displayed on the image (block 403) through, for example, guidance module 740, enabling the user to align the hysteroscope with the recommended entry trajectory. The hysteroscope may be aligned with the recommended entry trajectory and inserted into the cervix. At block 404, the cervical canal may be visualized, for example using an imaging sensor of the hysteroscope or another sensor. At block 405, a center of the cervical canal may be determined. Using this displayed center point at block 406, the hysteroscope may be inserted into the cervical canal (block 407) and advanced while the canal lumen is visualized (block 408), using near field and/or far field imaging. As the view changes, the system performs additional image analysis to determine an updated center of the cervical canal (blocks 404-406) and presents the center of the cervical canal as a visual marker on the image (block 407). The hysteroscope may be advanced through the cervical canal in accordance with the displayed canal-center indicator until the system determines that the tip of the hysteroscope has entered the uterine cavity.

Once inside the cavity, the system enables far-view and near-view imaging (block 408) so that broader regions and finer-detail regions of the endometrial surface can be captured (block 409). The system then combines the far-view and near-view data to generate a three-dimensional endometrial surface and cervical canal map (block 410) using modules such as the 2D/3D model generation module 750, which may incorporate any of the computer vision techniques disclosed herein. The system may determine whether the endometrial surface has been adequately mapped (block 409), and if not, prompts additional imaging until sufficient coverage has been obtained. During mapping or after mapping, the system may calculate one or more endometrial and cervical measurements (block 411), including dimensions, angulations, anatomical relationships, and other parameters, based on the reconstructed models. These outputs may be displayed to the user through graphical overlays, numeric fields, or other user-interface elements generated by the guidance module 740. The hysteroscope may be optionally removed (block 412) and/or a working device inserted and/or navigated using hysteroscopy-generated maps / measurements (block 413).

FIG. 4B shows an embodiment of method of performing a subsequent procedure after mapping one or more portions of the endometrial surface. The method may use previously acquired anatomical mapping information to guide subsequent intrauterine navigation and interventional procedures. After an initial uterine cavity map has been generated, the method may optionally include navigating a hysteroscope within the endometrial cavity (block 414) using visual cues, overlays, and/or guidance produced by system 700. In some embodiments, as the hysteroscope is navigated, the system actively maps the endometrial surface (block 415), continuously updating the anatomical reconstruction based on new image frames captured from hysteroscope 100. The system may evaluate whether the desired endometrial surface has been adequately mapped (block 416) by determining whether one or more regions of the cavity have been imaged with sufficient coverage. If the system determines that the desired surface is not yet fully mapped, the system may provide feedback on the remaining unmapped or inadequately mapped regions (block 417), prompting the user to reposition the hysteroscope to obtain additional imaging of those areas. If the system determines that the desired surface is not yet fully mapped due to debris blocking the view, the system may convey information about the location of the remaining unmapped or inadequately mapped regions, and a debris removal device may be used to remove the debris, such as a suction device.

Once adequate coverage has been achieved, the system may generate a 3D endometrial surface and target anatomy/pathology map (block 418) using one or more reconstruction techniques described elsewhere in this disclosure, such as multi-view photometric methods, structure-from-motion, or computer-vision/AI-based reconstruction using modules such as 750 and 794. From the cavity map, the system proceeds to generate endometrial and cervical canal measurements (block 419), including dimensional, angular, and/or spatial parameters relevant for diagnosis, documentation, or procedural planning.

With the mapping and measurements complete, optionally, a working device 102 may be introduced, and the system may track the device in real time during its movements within the cavity (block 420), using sensor-based tracking, image-based tracking, or a combination thereof. In some embodiments, simultaneously or near simultaneously, the system may display real-time procedural parameters (block 421), for example, device depth, angular orientation, trajectory alignment, target proximity, and/or warnings about anatomical risk zones, to support accurate and safe manipulation of the device. In some embodiments, updated images of the cavity may be acquired, and the system may generate a post-procedure 3D endometrial surface map (block 422) to document procedural outcomes such as resected regions, ablated zones, or cleared adhesions. Finally, the system may generate a post-procedure report (block 423) that includes the updated anatomical map, measurements, procedural parameters, pathology characterizations, and other clinically meaningful outputs, as described elsewhere in this specification.

FIG. 4C shows an embodiment of a method 301 of generating clinical information using one or more deep learning models. At step 324, an imaging device may be used to scan a patient. Examples of imaging devices include, but are not limited to: DXA scanners, X-ray imaging devices, MRI scanners, CT (including micro-CT) scanners, ultrasound scanners, and any of the devices disclosed herein. At step 326, imaging data may be obtained from the imaging device. At block 328, the imaging data may be pre-processed. Examples of pre-processing steps and techniques are discussed elsewhere in this specification. They include, but are not limited to: cropping one or more images, segmenting the one or more images based on one or more parameters, feature extraction, etc.

Segmentation or block definition may be performed in any of the images disclosed herein. Segmentation may be performed manually or using computer-based methods (e.g., using convolutional neural networks like U-Net). Examples of segments include, but are not limited to: bone of interest, trabecular bone, cortical bone, bone margins, soft tissue, regions with similar bone mineral density levels, regions with similar radio-opacity levels, etc. Any of the analyses disclosed herein may be performed on individual segments, or combination of segments. In some embodiments, one or more analyses disclosed herein may be performed solely on trabecular bone. In some embodiments, one or more analyses disclosed herein may be performed solely on cortical bone. In some embodiments, one or more analyses disclosed herein may be performed solely on soft tissue. Examples of segmentation methods include, but are not limited to:
1. Object-oriented approaches comprising several stages starting with the most general objects to be segmented, such as bone outlines from a background, and proceeding in a succession of stages to the most specific object(s), such as trabecular bone. Each stage may use custom operators unique to the needs of that specific stage for increased accuracy.
2. Approaches using deep leaning. For example, using convolutional neural networks (CNNs) such as SDResU-Net, MD-LSTM, U-Net, RFCN, 2D CNN, DeepLab, FCN, etc.
3. Entropy-based segmentation techniques
4. Region-based algorithms in which the regions are constructed using a growing procedure with two or more statistical tests. Tissue classification procedures may be employed following the growing process.
5. Phase based atlas registration
6. Thresholding. This method separates regions in an image based on intensity values above or below a specified threshold.
7. Active contours techniques. In these techniques, deformable models may be used that evolve to fit object boundaries by minimizing an energy functional.
8. Watershed Segmentation wherein an image is treated as a topographic map and segmented based on watershed lines.
9. Methods that track evolving interfaces over time
10. Clustering methods
11. Graph-Cut Segmentation wherein an image is modeled as a graph and segmented by locating the optimal cut based on pixel affinities.
12. Markov Random Fields
13. Atlas-Based Segmentation which involve registering a template or atlas to the target image and then transferring the segmented regions from the template to the target.

The pre-processed data may be fed to one or more artificial intelligence-based methods such as deep learning or other machine learning (ML) models. In several embodiments of this invention, machine learning techniques are used examples of artificial intelligence-based methods.

However other artificial intelligence-based methods such as computer vision-based algorithms, image segmentation algorithms, Fourier transforms and other transforms, fuzzy logic-based methods may also be used. Such methods may be used in conjunction with machine learning. In this embodiment, the data may be sent to three ML models. The output from one or more ML Models may then be post-processed as shown in step 336. Examples of post-processing steps include, but are not limited to: displaying one or more output data superimposed on one or more input data, highlighting the areas of new bone growth, highlighting the areas of new bone loss, etc. At block 338, clinical information may be generated. This may be done by combining the output of any of the methods and devices disclosed herein with one or more additional clinical data. Examples of clinical data are disclosed elsewhere in this specification, but may include, but are not limited to: patient preference, clinical background data, radiologic data (which may be obtained from one or more device and/or methods disclosed herein), biochemical markers, lifestyle data, wearables data, sensor data, drug(s) data, therapy(ies) data, etc.

In any of the embodiments disclosed herein, a machine learning model may be used to perform one or more tasks, examples of which include, but are not limited to: predicting the treatment outcomes of one or more treatments and/or natural clinical course of the patient without interventions. Such models may be used in any suitable system or method embodiments disclosed herein. Such models may be built or trained using one or more images and one or more clinical data. Several examples of imaging modalities used to generate the images are disclosed elsewhere in this specification. They include radiological modalities such as DXA, X-rays, MRI, CT, ultrasound, etc. Examples of clinical data are also disclosed elsewhere in this specification. Clinical data e.g., clinical background data, biochemical marker(s) data, lifestyle data, wearables data, sensor data, drug(s) data, therapy(ies) data, etc. may be used as metadata for the images used to build or train the ML models. Examples of methods used to build such ML models include, but are not limited to:
1. Convolutional Neural Networks (CNNs): CNNs can be used to automatically extracting features relevant to the prediction task. Examples of CNNs include, but are not limited to: AlexNet, VGGNet, ResNet, and Inception. Metadata may be incorporated into CNN models through one or more methods such as fusion with image features, parallel processing pipelines, etc.
2. Vision Transformer (ViT) model architectures that leverage the transformer model.
3. Deep neural networks, wherein regions of interest (ROIs) are extracted from an image by a rotated object detection technique used in region proposal networks.
4. Deep neural networks, wherein anatomical landmarks are detected and used for extracting ROIs. One or more calculations and functions disclosed herein may be performed on the extracted ROIs.
5. Prediction models for predicting one or more bone parameters such as bone strength, bone density, bone growth, etc. based on deep convolutional neural networks.
6. Graph Neural Networks (GNNs): GNNs may be used to model relationships between patients based on metadata (e.g., patient similarity graphs). Image data may be incorporated as node features.
7. Deep Learning Architectures using Multi-Modal Fusion: Multi-Modal Neural Networks (MMNNs) or multi-input and/or multi-output networks may be used to learn joint representations of image and metadata data, thereby allowing for multiple interactions between such data.
8. Transfer Learning: In such methods, pre-trained deep learning models (e.g., CNNs) may be fine-tuned on medical image datasets along with patient metadata. In transfer learning methods, knowledge from large datasets may be used to improve performance on smaller, domain-specific datasets.
9. Support Vector Machines (SVMs): Extracted features from both images and metadata may be used as inputs to SVM models. SVMs may be used for classification tasks.
10. Random Forests and Gradient Boosting Machines: Ensemble learning methods like Random Forests and Gradient Boosting Machines may be used to process multiple data types and capture complex interactions between features. Such methods may incorporate image features extracted using techniques such as histogram of gradients as well as features of metadata.
11. Recurrent Neural Networks (RNNs): RNNs can be used for sequential data modeling, such as time-series data or sequential patient records. Metadata can be treated as sequential data (e.g., temporal changes in patient vitals), while images may be processed using techniques such as CNNs.
12. Ensemble models may be used to perform one or more classifications and or calculations disclosed herein. In such ensemble models one or more additional inputs may be provided. Examples of such additional inputs include, but are not limited to: details of the drug therapy being administered to the patient, bone mineral density data, other imaging data, the patient demographic data, data on the size and other parameters of the organ or tissue being imaged, data obtained through a digital health or consumer device, etc. Details of the drug therapy include but are not limited to: type of drug, route of administration, dosage, dosage history, duration of treatment, date(s) of Initiation of treatment, date(s) of completion of treatment, etc.
13. Hybrid Approaches: One or more of the above methods may be combined. E.g., CNNs may be combined with RNNs for processing sequential data along with images.

Any of the ML models disclosed herein may be trained using one or more clinical endpoints or labels including, but not limited to: presence or absence of osteoporosis, fracture risk, presence or absence of crack propagation sites, BMD, response to therapy, true bone strength (which may be calculated e.g.,, through Finite Element Analysis-FEA), bone quality, trabecular score, bone strength parameter(s) determined using human cadaver bones, etc. Bone strength testing (e.g., resistance to impact, resistance to crack propagation) or other tests may be performed on cadaver bones, and the data may be fed to or otherwise used by one or more device or method embodiments herein. The training may be performed using supervised machine learning, wherein labeled datasets are used to train algorithms that classify data or predict outcomes. After input data is fed into a model, the model may adjust its weights until the model has been fitted appropriately as determined through a cross-validation process. These clinical endpoints may be determined by a computer-based or human analysis and used to label one or more training datasets. In one such example, one or more processed images (examples include, but are not limited to: images taken at baseline, images taken after administering a therapy, images showing a change relative to time, etc.) are fed to a ML model along with one or more clinical endpoints (labels) such as bone strength. The bone strength may be determined through methods including, but not limited to: CT scanning, MRI scanning, bone biopsy analysis, FEA, etc. The ML model is then trained to classify and/or score one or more input images to determine actual or predicted changes in bone strength.

In some embodiments, the system identifies two or more regions of interest of the imaged tissue (e.g., one or more regions of one or more bones). The system performs data processing on two or more regions of interest using two or more methods or models. In such embodiments, a first machine learning model (e.g., ResNet) may be used to analyze a cortical region of interest and a second machine learning model (e.g., EfficientNet) may be used to analyze a trabecular region of interest.

For any of the machine learning models disclosed herein, a Multiple-blocks regression strategy may be used. In this strategy, the image is divided into multiple blocks. This may be done using anatomical markers and/or measurements and/or segments. The one or more blocks may be one-hot-encoded and further processing may be performed.

FIG. 4D shows an example of a flow chart of a supervised ML model that uses labeled data. Training data 340 is cleaned or otherwise pro-processed and fed to a feature extraction module to extract one or more useful extracted feature(s) 342 from the data. The one or more labels 344 or known class along with extracted feature(s) 342 may be passed to the training phase where machine learning algorithms 346 may be used to identify a particular model which map inputs to desired outputs. Model evaluation 350 may be used to provide feedback to feature extraction and learning phases. The extracted feature(s) 342 and/or ML algorithm(s) may be adjusted to improve accuracy of ML model 348. The training process may be repeated one or more times until a desired accuracy level is achieved. The ML model 348 may then be used to perform one or more methods disclosed herein, e.g., to predict the labels of data obtained from an imaging device.

In some embodiments, ML model 348 may be trained using endoscopy images and/or sensor data, examples of which are disclosed elsewhere herein. Labels 344 may include one or more of: fertility parameters, endometrial thickness measurement(s), presence/absence of malignancy or hyperplasia, presence/absence of adenomyosis, response to specific drugs or therapies, etc. Trained ML models 348 may be used for predicting such data based on input from an endoscope. One advantage of such embodiments is the ability to immediately determine or predict parameters such as fertility parameters, endometrial thickness measurement(s), etc. This allows the user to immediately take clinical decisions without waiting for additional testing.

In some embodiments, a trained ML model may be used for predictive analytics to predict the response of a patient to one or more drug or non-drug therapies. In some embodiments, a trained ML model may be used for predictive analytics to predict the response of a treatment-naïve patient to bisphosphonate therapy. A predicted lack of sufficient therapeutic response to bisphosphonate therapy may be used to treat the patient using non-bisphosphonate therapies; examples of which are disclosed herein. In another embodiment, a trained ML model may be used for predictive analytics to predict the further response of a patient to an ongoing therapy. A predicted lack of sufficient continuing therapeutic response may be used to switch the patient to another therapy; several examples of which are disclosed herein.

FIGS. 4E and 4F show two method embodiments showing how an AI-based model of the present disclosure may be incorporated in a treatment method using an imaging system. In the method shown in FIG. 3D, clinical inputs at block 500 are fed to an imaging system at block 510 incorporating an AI-based model at block 512. Examples of imaging systems and AI-based models are disclosed elsewhere herein. The imaging system then produces one or more outputs at block 514; examples of which are given elsewhere in this specification. In some embodiments, clinical inputs may be fed to an imaging system. The output of the imaging system may be fed into one or more AI-based models. Examples of imaging systems and AI-based models are disclosed elsewhere herein. The AI-based model may generate one or more outputs, examples of which are given elsewhere herein.

Any of the outputs or other data generated by the embodiments herein may be used as inputs for subsequent analysis. Outputs at block 514 can be any of the outputs of any of the embodiments herein. Examples of outputs at block 514 include, but are not limited to: anatomical data or maps (examples shown in FIGS. 1K, 3L-3N, 3P-3S, etc.), anatomical measurements (examples shown in FIGS. 2I, 3B, 3C, etc.), and reports containing text and/or graphics (example shown in FIG. 3M, etc.). Examples of such analyses include, but are not limited to: analyses performed using artificial intelligence methods (e.g.,, those using applications or AI models based on foundation models, computer vision / AI module 794), analyses performed using AI agents or copilots, analyses performed using machine learning models, analyses performed using predictive models for predicting outcomes such as safety and efficacy outcomes, comparative analyses performed using the same or similar outputs at two or more timepoints, etc. Foundation model types include, but are not limited to, large language models, vision models, multi modal models, etc. Any of the outputs or other data generated by the embodiments herein may be used as inputs for performing subsequent interventions such as diagnoses or therapies. Examples of such subsequent interventions include, but are not limited to: guiding one or more devices within the anatomy, selecting one or more surgeries or procedures for the patient, selecting the timing(s) and/or dose(s) of administration of one or more drugs or other therapies, guiding one or more surgeries or procedures, selecting one or more parameters of one or more surgeries or procedures, predicting clinical parameters such as fertility, etc.

FIG. 4G shows an embodiment of one such method. One or more outputs at block 514 are fed to a processing system. Additional data may be obtained as shown in block 328. One or more analyses may be performed, as described elsewhere herein. Surgical planning model(s) 330 may be used to plan one or more steps of a subsequent surgical procedure. Some examples of such surgical procedures are listed elsewhere herein. Information may be extracted from the data fed to the processing system as shown in block 331. The extracted information may be fed to a diagnosis model 332 which diagnoses one or more conditions. Some examples of such conditions are listed elsewhere herein. Disease prognosis model 334 may be used to determine the natural or other prognosis of one or more conditions or diseases. Some examples of such more conditions or diseases are listed elsewhere herein. Data or results from such analyses may be post-processed. The method of FIG. 4G may generate final output(s) or clinical Information at block 338.

In some embodiments, multi-modal and multi-scale data fusion techniques may be used to generate an integrated view of the anatomy, physiology, pathology, etc. of a patient. Multi-scale data is defined as data represented and/or analyzed at multiple levels of resolution, granularity, or scale such as time, space, frequency, or abstraction. Such multi-modal and multi-scale data may be used to guide one or more of: insertion, navigation, and/or placement of any of the devices disclosed herein. FIG. 4H shows an example of a method that uses multi-modal and data fusion techniques. In the example shown, hysteroscopic data 360 (examples of which are described herein) undergoes one or more feature extraction step(s) to generate a feature vector database 342 of hysteroscopic data. Data from other modalities (e.g., ultrasound data 362) may undergo one or more feature extraction step(s) to generate a feature vector database 364 of data from that modality. Feature vector databases (e.g., databases 342 and 364) may be combined into database 368. Clinical data 366 may be integrated into database 368 through data integration techniques.

In some embodiments, feature extraction (e.g., using feature extraction module 754) may be performed using encoder-decoder models. Such models may be used for segmentation, denoising, classification, and reconstruction in any of the embodiments described herein. One or more encoders may compress an input image into a lower-dimensional representation (e.g., feature map). This may be used to capture high-level features such as shapes, textures, and spatial patterns. In some embodiments, one or more decoders may be used to reconstruct the image or output a task-specific result (e.g., segmentation map). In any embodiment described herein, such models may be used for tasks including, but not limited to:

Segmentation (e.g., of anatomical regions, pathologies, etc.). In such embodiments, one or more features from the encoder layers may represent learned spatial and contextual information.

Classification (e.g., normal vs. pathological tissue). In such embodiments, one or more encoder features may be fed into a classifier.

Anomaly Detection. In such embodiments, one or more autoencoders may learn to reconstruct normal images such that deviations indicate anomalies.

Modality Translation. In such embodiments, image-to-image translation may be performed (e.g., ultrasound or MRI data to hysteroscopic data).

Encoder-decoder models may be constructed using architectures such as U-Net, Autoencoders, Variational Autoencoders (VAE), Transformers, etc.

FIG. 4I shows an example of a method of training a Large Language Model (LLM) and using such trained LLMs for clinical applications. At block 370, hysteroscopy is performed. At block 372, imaging and/or clinical outputs are generated; several examples of which are described herein. At block 374, data such as imaging and/or clinical outputs are analyzed using vision models. At block 376, one or more of: structural information, measurement(s), and abnormality data is generated. Several examples of such methods and data are described herein. At block 378, an LLM is trained using one or more of: structural information, measurement(s), and abnormality data. At block 380, the trained LLM is queried using data from a patient such as imaging data, clinical information, etc. At optional block 382, the LLM may be used to generate a clinical report based on the query of block 380.

Any of the outputs from the embodiments disclosed herein may be used to develop vision-language medical models which combine imaging and text data. Such models may be used for automated hysteroscopic report generation.

Convoluted Neural Networks (CNNs) may be used to detect patterns like edges, shapes, textures, density differences, etc. in any of the outputs from the embodiments disclosed herein. Vision Transformers (ViTs) may be used for detection of subtle lesions, identify complex spatial patterns, and relationships between various imaging findings. Any of the output data described herein may be used as input for an LLM layer. In some embodiments, one or more imaging models may be used to identify findings, and an LLM may be used to process them in context. LLMs may be used for language generation, reasoning with medical knowledge, and/or synthesizing multiple data sources. LLMs may be used to convert findings into radiology reports by combining image findings with other data (e.g., symptoms, medical history, surgical history, lab results, risk factors). As an example, an imaging output (e.g., reconstruction of uterine cavity) could be used as an input into an LLM which could generate a report specific to the patient after analyzing other data sources, such as medical records, medical/surgical history, symptoms, background information, etc.

In some embodiments, for example, as part of computer Vision / AI module 794, a multimodal LLM that analyze images may be built using paired image-text data. The text may be converted into tokens, while images may be converted into numerical representations, e.g., by splitting them into patches or encoding them into feature embeddings. Such systems may have two main components: a vision encoder and a language model. The vision encoder, such as a convolutional neural network or vision transformer, may be used to processes the image and outputs embeddings. The language model (e.g., based on a transformer architecture) may be used to process text and generate outputs. Aligning the image and text representations may be achieved by training the model to associate visual features with corresponding language concepts so that similar patterns in images map to consistent textual meanings. The models may then be trained on multimodal tasks such as image captioning, visual question answering, and report generation. This may enable the model to reason across both image and text inputs. The models may be fine-tuned for specific applications and evaluated. In deployment, images may be processed by the vision encoder, combined with text representations, and interpreted by the language model to produce outputs such as descriptions, diagnoses, answers to queries, or reports.

One or more sensors, for example, sensor(s) 760, may be incorporated into any of the devices disclosed herein such as hysteroscope 100, a device connected to hysteroscope 100, etc. Examples of such sensors include, but are not limited to: image sensors (e.g., Complementary Metal Oxide Semiconductor (CMOS) sensors, Charge-Coupled Device (CCD) sensors, etc.), light sensors, accelerometers, gyroscopes, pressure sensors, temperature sensors, impedance sensors, electrophysiological signal sensors, ultrasound or other visualization elements, flow sensors, motion sensors, Light Detection and Ranging (LiDAR) elements, sensors based on Doppler effect, etc. One or more sensors may be used to track movements of one or more devices disclosed herein. This movement/positioning data may be used for determining orientation relative to anatomy, documentation, training, guiding subsequent procedures, alerting user or correct or incorrect use, alerting user of risks, obtaining imaging coverage of the entire cavity surface, etc. Various embodiments of orientation/location determining mechanisms that track the orientation/location of the camera/distal tip of the hysteroscope or other devices in real time may be present on embodiments described herein. Such orientation/location determining mechanisms may be located on devices connected to hysteroscope 100, working device 102, or elements connected to such devices. In some embodiments, gyroscopes and/or accelerometers may be used to tracks the twisting and/or translation of hysteroscope 100 or other devices described herein.

As shown in FIG. 7, the systems and methods of the embodiments disclosed herein can be embodied and/or implemented at least in part as a machine or controller for receiving a computer-readable medium storing computer-readable instructions. The machine or controller may be configured to receive an imaging output of the endoscope. The imaging output may be a video stream generated by the endoscope, computer-processed data from a video stream generated by the endoscope, etc. Such machines or controllers may comprise a processor 762 and a memory 764 storing instructions that can be executed by the processor 762 (e.g., Central Processing Unit, Graphics Processing Unit, Tensor Processing Unit, Digital Signal Processor, Field-Programmable Gate Array, Application-Specific Integrated Circuit, System-on-Chip Architecture, Cloud or Server Based Processing Cluster, etc.) and one or more trained artificial intelligence models (e.g., computer Vision / AI module 794). The instructions may be executed by computer-executable components preferably integrated with the system and one or more portions of the processor on the scanner and/or computing device. The computer-readable medium can be stored on any suitable computer-readable media such as RAMs, ROMs, flash memory, EEPROMs, optical devices (e.g., CD or DVD), hard drives, floppy drives, or any suitable device. In some embodiments, the computer-readable medium is in electronic communication with an imaging device such as an endoscopy device. In some embodiments, the computer-readable medium is a component of an imaging device. The computer-readable medium may store information from current and prior scans. For example, the computer-readable medium may store information about prior scans such that the data from subsequent imaging can be compared to prior imaging. The computer-executable component is preferably a general or application-specific processor, but any suitable dedicated hardware or hardware/firmware combination can alternatively or additionally execute the instructions. The computer-executable component, an example of which is shown in FIG. 3C, may be designed to allow parallel processing using a processor, for example a Graphics Processing Units (GPUs). Other exemplary processors may be used such as a Field-Programmable Gate Array, an Application-Specific Integrated Circuits, and the like. The system may use a computer-executable component, one or more computer-executable components, or a plurality of computer-executable components on a computing device, one or more computing device, or a plurality of computing devices. The computing devices may be local, remote, a server, in the Cloud, workstation, etc. The one or more computing devices may be networked, in some embodiments.

Any of the embodiments disclosed herein may be used in autonomous or semi-autonomous imaging systems. Any of the embodiments disclosed herein may comprise self-navigating functionality wherein the hysteroscope will determine the direction of a lumen and will modify itself accordingly to reduce force, pressure, or pain on the walls of the surrounding anatomy. Any of the embodiments disclosed herein may be used in robotic systems (e.g., robotic navigation system 710). Any of the systems or their components (e.g., hysteroscope 100, working device 102, hysteroscope channel 104, etc.) disclosed herein may be navigated and/or positioned and/or controlled by one or more mechanisms. Examples of such mechanisms include, but are not limited to: pull-wire or cable-driven mechanism(s), gear and rack-pinion mechanism(s), tendon-sheath mechanism(s), mechanism(s) based on shape-memory alloys and smart materials, hydraulic and/or pneumatic actuation mechanism(s), etc. Any of the embodiments of robotic systems may be controlled using one or more methods; examples of which include, but are not limited to: teleoperation (e.g., leader-follower control or command-response control), haptic feedback, AI and/or Machine Learning-based control (e.g., using algorithms for decision-making, movement prediction, etc.), computer vision and image-guided control, autonomous or semi-autonomous control, etc. One or more method steps (e.g., patient positioning, endoscopic scanning, quality checking, report making, etc.) may be performed using systems that have a degree of autonomy. Pretrained models may be used to perform and/or assist any of the method steps disclosed herein. In one such embodiment, one or more AI models fine-tuned for robotics may be used for one or more of: obtaining imaging data, analyzing imaging data, performing one or more tasks, etc. using vision and/or language processing. In one such embodiment, one or more AI models fine-tuned for imaging may be used for one or more of: obtaining imaging data, analyzing imaging data, providing one or more outputs or commands, etc. using vision and/or language processing. In some embodiments, imaging information and/or one or more outputs obtained from one or more embodiments herein may be used to perform any method steps (e.g., movement(s), steering(s), adjustment(s), guiding(s), etc.) of any embodiment herein in an autonomous or semi-autonomous mode wherein the user does not provide an input for that method step.

One or more devices or methods (e.g., increasing distension fluid pressure or flow) may be used to remove fluffy tissue or floating debris to improve visualization. In some embodiments, the system may temporarily increase the rate or pressure of distension fluid flow to generate a flushing effect within the uterine cavity, thereby mobilizing and clearing loose endometrial fragments, mucus strands, blood clots, or other particulate material that may obscure the FOV. In other embodiments, a dedicated working device, such as a suction catheter, irrigation-aspiration instrument, or a mechanical debris-removal element, may be introduced through hysteroscope channel 104 to actively dislodge or remove such debris. The system may automatically determine when visualization is degraded based on image-quality metrics (e.g., brightness inconsistency, visual occlusion patterns, or reduced feature detectability) and may prompt the user or autonomously initiate a debris-clearing maneuver. In some embodiments, the system may modulate fluid flow direction, pulse frequency, or suction settings in a closed-loop manner to efficiently clear the field while avoiding excessive force on the surrounding anatomy. These debris management features enable the autonomous or semi-autonomous imaging system to maintain optimal visualization, thereby supporting more reliable navigation, mapping, and device guidance performance during subsequent steps of the procedure.

Distension fluid pressure may be automatically controlled by a system. In one such embodiment, closed loop feedback based on the adequacy of uterine cavity distention is used to automatically control distension fluid pressure. The adequacy of uterine cavity distention may be calculated by the system based on one or more parameters, examples of which include, but are not limited to: visualization of one or more anatomical structures like tubal ostia, distance(s) between uterine and/or cervical walls, response of the uterine and/or cervical wall to pressure or flow changes, presence of debris or other obstructions in the visual field, etc.

In some embodiments, one or more access devices such as guidewires or catheters may be introduced through a hysteroscope. The hysteroscope may be removed leaving the access device in place. The access device may then be used to introduce one or more intrauterine or intrafallopian tube devices.

Any of the method and device embodiments disclosed herein may be loaded/attached/connected or otherwise incorporated into a hysteroscopy devices or systems.

Any of the method and device embodiments disclosed herein may be used for one or more applications, examples of which are listed in the following categories:

1. In General Gynecology

For calculating/predicting the sounding length (SL) and cavity length (CL). SL or CL may be predicted when a rigid instrument (e.g., a rigid sound) is used. SL or CL may be predicted when a flexible instrument (e.g., a flexible sound) is used.

For calculating uterine cavity width and length automatically.

For diagnosing and/or treating conditions such as endometritis hyperplasia PCOD/PCOS Niche in the Cesarean Scar (Isthmocele).

For determining menstrual cycle phase.

For calculating endometrial cancer risk.

For determining post-menopausal status.

For calculating/estimating endometrial thickness at one or more locations around the uterine cavity.

For detecting/calculating/estimating uterine peristalsis or other muscle movement(s). This in turn may be used to diagnose conditions such as adenomyosis, etc. For collecting tissue from a first suspected area, collecting tissue from a second non-suspected area and comparing the results.

For highlighting red spots/regions on hysteroscopy images due to bleeding.

For correlating ultrasound images with data from hysteroscopy images. This may be used to perform virtual hysteroscopy.

For assessing relative movement of tissue when the tissue is subjected to fluid flow, mechanical manipulation, etc. This may be used to differentiate polyps, adhesions, type 0 fibroids, polypoid endometrium, etc.

For marking the cycle time in a visual format, e.g., regular ovulatory cycles, days post bleeding, anovulatory cycles, etc.

For identifying the orientation of the cornu opening. Thereafter, one or more devices/hysteroscope 100 tips may be oriented or moved to allow them to pass through the opening. This may be used for contraception, fallopian tube patency check, opening fallopian tubes, salpingectomy, etc.

In some embodiments, the 3D profile of the uterine cavity surface may be converted from a distended profile (as seen by the hysteroscope) to a non-distended profile. Any of the images or other outputs described herein may be produced with the non-distended profile of the uterine cavity. Examples of such images or outputs include but are not limited to those illustrated in FIGS. 3A-3Q, etc.

For identification / classification of pathology. e.g., for automatic size estimation of anatomy, pathology, landmarks, etc.

For generating a 3D map of the uterine cavity and/or cervical canal. In some embodiments, 3D maps of the uterine cavity at two different times may be compared by processing one or both maps to account for differences in the distension pressure.

For generating an annotated map of the uterus.

External visual markings on the hysteroscope 100 or any other device(s) described herein may be used to guide hysteroscope placement and/or movements and/or insertion depth into a structure. In some embodiments, any of the methods described herein may be performed to determine parameters such as sounding length, uterine cavity length, uterine cavity width, etc. Thereafter, one or more insertion depth parameters (e.g., maximum insertion depth, minimum insertion depth, etc.) of a hysteroscope 100 or any other device(s) may be determined. Examples of devices include, but are not limited to: biopsy devices, curettage devices, needles and other injecting devices, sampling devices, insemination devices, tissue harvesting devices, material depositing devices, IUDs and their insertion devices, and other examples disclosed herein. Such hysteroscopes 100 or other device(s) may then be inserted using the relative positions of the external markings and the anatomy (e.g., external cervical os) as a guide.

In one embodiment, hysteroscopic assessments are performed while withdrawing the hysteroscope 100. Hysteroscope 100 may be moved to visualize the entire uterine cavity. If adequate imaging coverage is not obtained, the user may be prompted and more imaging performed.

For dilatation and curettage: The uterine anatomy may be checked and/or measured for guiding the placement / introduction / movements of a curetting device.

For identification / display of sites of injecting anesthetics or other agents for procedures such as paracervical block, intracervical block, fundal block, etc. in uterine cavity regions (e.g., fundal regions, cornual regions, etc.), intra-cervical regions, cornual regions for reducing pain, preventing spasm, etc. In some embodiments, one or more sites of injecting may be determined based on one or more factors; examples of which include, but are not limited to: location(s) of pathologies such as fibroids, uterine cavity shape, uterine cavity size parameter(s), uterine innervation, etc.

For navigation through the uterine cavity or cervical canal or fallopian tubes based on the visuals. For example, a hysteroscope tip may be bent or adjusted for easier, less painful, and/or less risky insertion.

For injecting anesthetic for diagnosing uterine pain conditions such as dysmenorrhea and adenomyosis. If the pain goes away or reduces after injection, it may mean that the patient is suffering from a uterine pain condition.

For automatic adjustment of the lighting (e.g., intensity of one or more illumination source(s), type of illumination sources in operation, etc.) to achieve optimal image quality. Optimal image quality may be achieved by preventing under illumination or over illumination, preventing image washout in certain areas, etc.

2. In Infertility Evaluations and/or Treatments

To check tubal patency by visualizing flow.

For diagnosing and/or treating polyps, myoma, etc.

For diagnosing and/or treating proximal tubal block through cannulation.

For diagnosing and/or treating Müllerian anomalies, T-shaped uteri, tubular-shaped uteri, uterine dysmorphism.

For determining one or more fertility affecting factors including, but not limited to: endometrial receptivity, endometrial health, endometrial status, cycle phase, implantation receptivity, endometrial texture, etc.

For determining defective endometrial receptivity and/or poor reproductive performance, especially due to uterine malformations.

For visualizing endometrium before in vitro fertilization (IVF).

For generating data to be used with laparoscopy-generated data.

For determining endometrium response to fertility drugs.

For fertility status/quantification.

For determining best sites and/or time point of implantation.

For determining best sites or time point of Intrauterine insemination (IUI).

For determining best target location for infertility interventions.

For checking patency of fallopian tubes.

For achieving patency of tubes using devices or fluids. This may be followed by checking if patency has been achieved.

For generating input(s) for fertility treatments such as treatment parameters, expected success rate, treatment selection, etc.

3. In Abnormal Uterine Bleeding Evaluations and/or Treatments

For diagnosing and/or treating polyps and myoma.

For diagnosing and/or treating dysfunctional uterine bleeding (DUB).

For endometrial ablation procedures. During endometrial ablation procedures, one or more parameters or outputs generated from any of the methods disclosed herein may be used for one or more of: mapping the uterine cavity, determining suitability of the cavity for endometrial ablation, determining insertion depth of a device, determining uterine cavity length and/or width, modulating energy output of a generator, determining one or more parameters (e.g., gas flow rate, pressure threshold, etc.) of cavity integrity checks (checks to detect perforation), detecting uterine perforation or false passage, determining expected width of the ablation device after deployment, setting the deployed length and/or width of an active end of an ablation device, determining the position of cervical shields or other device elements to protect the cervix, determining one or more operational parameters of endometrial ablation procedures, predicting one or more operational parameters of endometrial ablation procedures, modulating one or more operational parameters of endometrial ablation procedures, etc. Examples of operational parameters of endometrial ablation procedures include, but are not limited to: power level(s) during energy delivery, power limit, frequency of operation, impedance levels, impedance limits, energy delivery time, time limits, suction pressure during operation, ablation depth(s), deployment location(s), energy dose(s), parameter of pre-procedure checks, operational parameters of one or more sensors located on an endometrial ablation device, returned power limits, etc.

4. In Ashermann’s Syndrome

For treating amenorrhea.

For (re)creating a cavity by breaking adhesions.

For detecting and/or quantifying and/or classifying adhesions.

5. For Contraception Methods

For guising the insertion and/or navigation and/or deployment of trans-cervical tubal sterilization and other sterilization methods.

For detecting and/or retrieving misplaced intrauterine devices.

For customizing an IUD based on measured uterine dimensions. Such IUDs may be placed based on measured insertion depth and other uterine parameters. An insertion device for inserting any IUDs disclosed herein and/or an IUD may be customized based on one or more measured uterine dimensions and other parameters.

For reducing pain during hysteroscopic IUD placements by guiding the navigation of the IUD insertion device based on prior imaging or measurements.

For adjusting/calculating a drug dose (e.g., copper/progestin in an IUD) based on one or more measured and/or calculated uterine parameters like uterine cavity surface area, endometrial volume, etc.

For guided placement of IUDs. In one such embodiment, the uterus is imaged and an intrauterine device is selected based on the imaging, an access path is determined into the uterine cavity, and an IUD is inserted using the access path into the uterine cavity.

For checking contours of uterine walls to select IUDs or for insertion/placement method step(s) determination.

For selecting the type of IUD based on uterine cavity or cervical anatomy.

For determining site of implanting a frameless IUD. Such an IUD may be implanted under visual guidance.

For guided application of tubal blocking agents such as Fembloc™. Guidance may be provided for one or more of: aligning with cornua, determining location/extent of endometrium into the fallopian tubes, positioning cannula/device tip, determining the site of injection of an agent, determining the site of introduction of an agent or device, etc.

For inserting intra-fallopian tube devices into a fallopian tube lumen. In some embodiments, the intra-fallopian tube devices may comprise one or more marker(s) such as visual markers to guide the insertion into the fallopian tube lumen wherein the insertion depth is determined by one or more embodiments herein. The insertion depth may be customized for each patient.

6. In Oncology-related Evaluations and/or Treatments

For diagnosis and treatment of endometrial carcinoma or other genital tract neoplasms.

For targeted biopsy.

7. In Obstetrics

For diagnosis and treatment of cervical ectopic pregnancy, isthmocele, RPOC, etc.

For embryoscopy.

8. In Evaluations and/or Treatments of Recurrent Pregnancy Loss

To treat Müllerian anomalies, e.g., resection of uterine septum.

To break synechiae.

In any of the abovementioned categories, artificial intelligence models may be trained using inputs (e.g. imaging data; some examples of which are described in this specification) combined with specific labels (e.g. endometrial thickness, menstrual cycle phase, endometritis, perforations, false passages, etc.). Methods such as the method shown in FIG. 4D may be used to train such models. In some embodiments, controllers or other capital equipment are designed such that they perform multiple functions. In some embodiments, such capital equipment may be designed to perform one or more diagnostic functions and one or more therapeutic functions. One such embodiment is shown in FIGS. 5A to 5E.

FIGS. 5A to 5E show various components of a system for performing diagnostic as well as therapeutic hysteroscopy. FIG. 5A shows an embodiment of a diagnostic hysteroscope comprising an elongate shaft 101 connected to a proximal hub 112, a proximal connector 114, a fluid inflow port 116, and a light port 118. An optional fluid outflow channel may also be present. The diameter of elongate shaft 101 may be low (e.g., less than about 4 mm) to enable easy insertion into the uterine cavity. Fluid inflow port is adapted to connect to a source of distension fluid (e.g., saline, contrast, gas, etc.). Light port 118 is adapted to connect to a suitable endoscopic light source e.g., high-intensity Xenon or light-emitting diode (LED) lamps through flexible optical connectors such as fiber-optic connectors. Hysteroscope 100 may also comprise a side arm 108 that contains the optical path. Side arm 108 terminates in a camera connector 110 that is adapted to connect to an optional external camera (not shown). The external camera may be used to generate images or videos that may be displayed on a screen of a controller 130 or an external display located on a video tower. The camera (e.g., with CMOS or CCD sensors) may comprise a software layer that can perform any of the processing functions disclosed herein. A camera control unit may be present to perform image processing steps such as adjusting color/white balance, edge enhancement, and brightness control. Hysteroscope 100 may be designed with a suitable viewing angle (e.g., about 0 degrees, about 12 degrees to about 15 degrees, about 30 degrees, etc.). Hysteroscope 100 may be designed with a rod lens for high quality visualization. In alternate embodiments, hysteroscope 100 may be designed with one or more image sensors 704 such as CMOS or CCD sensors, as shown in FIG. 7.

Even though, this embodiment shows an “integrated” design of hysteroscope 100 where multiple elements are combined in a single device, embodiments of hysteroscope 100 may be designed that have removable components such as outer sheaths containing fluid inflow port 116, inner sheaths containing a fluid outflow port, etc. In some embodiments, an outer sheath comprising fluid inflow port 116 may have a working channel.

FIG. 5B shows an embodiment of a working device that is designed to facilitate cavity visualization by removing soft debris or other floating or cavity-attached materials that obstruct visualization of the uterine cavity. In this embodiment, working device 102 comprises a shaft 120, a proximal hub 122, a proximal connector 124, and a debris removing element 126 that mechanically dislodges debris. Examples of debris removing elements 126 include, but are not limited to: rotating elements, reciprocating elements, suction elements, fluid delivery elements, mechanical scooping elements, etc. In method embodiments, debris removing element 126 may be used to remove debris without damaging and/or removing the endometrium. In some method embodiments, debris removing element 126 is used to remove debris that is blocking the view of one or more regions of the uterine cavity. Once such debris is removed, one or more diagnostic procedures (examples of which are disclosed herein) may be performed. In some embodiments, debris removing element 126 comprises a lumen through which gentle vacuum may be applied to evacuate the removed debris from the uterine cavity. In some embodiments, a gentle vacuum refers to a low-level negative pressure delivered through debris-removal element 126, sufficient to aspirate loose particulate material without disrupting uterine distension or causing trauma to the endometrium. Such gentle vacuum levels (e.g., about 70 mmHg to about 90 mmHg, up to about 150 mmHg, etc.) are substantially lower than those used for uterine evacuation procedures (which may exceed about 400 mmHg) and are regulated by the fluid-management or suction-control subsystem to maintain a stable visual field.

FIG. 5C shows an embodiment of an operative hysteroscope comprising an elongate shaft 101 connected to a proximal hub 112, a proximal connector 114, a fluid inflow port 116, and a light port 118. A working device 102 (e.g., working device 102 shown in FIG. 5D) may be inserted through a lumen of hysteroscope 100. Working device 102 may act as a fluid outflow channel. The diameter of elongate shaft 101 may be greater than the diameter of elongate shaft 101 of FIG. 5A to allow working device 102 to pass through hysteroscope 100. The functioning and use of fluid inflow port 116, light port 118, side arm 108, camera connector 110, etc. may be similar to those of the embodiment of FIG. 5A.

FIG. 5D shows an embodiment of a working device that is designed to remove cavity pathology such as fibroids and polyps. In this embodiment, working device 102 comprises a resecting mechanism 128. Resecting mechanism 128 may comprise rotating or reciprocating or other cutting elements to resect or cut cavity pathology. Removed material may be simultaneously suctioned out of the uterine cavity through a lumen of working device 102. In some embodiments, working device 102 is designed for mechanical morcellation (e.g., with a rotating and/or reciprocating blade located inside a windowed metal tube) of intrauterine pathologies. In some embodiments, working device 102 is designed for electro-mechanical morcellation of intrauterine pathologies. Multiple versions of working device 102 may be provided to the user. The user may select the appropriate working device 102 based on one or more analyses or outputs disclosed herein. Examples of such outputs include, but are not limited to: size/volume of the pathology, shape of the pathology, location of the pathology, type of the pathology, distance of the pathology from the fundus, distance of the pathology from the external cervical os, tissue boundaries, etc.

FIG. 5E shown an embodiment of a controller. Controller 130 may be used to control the functioning of hysteroscope 100 and/or working devices 102. Controller 130 may be used for functions such as delivering electrical or mechanical power to one or more working devices 102, providing a vacuum source to one or more working devices 102, etc. Controller 130 comprises an outer casing 132. Controller 130 comprises one or more device connectors 134 that are adapted to connect to devices such as working devices 102 shown in FIGS. 5B and 5D. Controller 130 may comprise one or more input elements 138 that select the mode of operation of controller 130. Depending on the mode selected, a suitable working device 102 may be connected to controller 130 through flexible connectors. Examples of modes include, but are not limited to: diagnostic mode, tissue debris removal mode, polypectomy mode, myomectomy mode, endometrial resection mode, fallopian tube access/cannulation mode, etc. Controller 130 may comprise one or more adjusting mechanisms 140 (e.g., dials, keypads, buttons, etc.) to adjust one or more parameters of operation of hysteroscope 100 and/or working devices 102. Controller 130 may comprise one or more displays 136 for displaying one or more data (e.g., video streams, timers, measurements, annotations, estimations, etc.) disclosed herein. Displays 136 may be touchscreens that collect one or more inputs, e.g., from the user. Controller 130 may comprise a foot switch connector. Such connectors may be connected to foot switches (e.g., pneumatic foot switches). In some embodiments. foot switches may be used to control the activation of working elements on working device 102.

In some embodiments, working devices 102 shown in FIGS. 5B and 5D are adapted to connect to a single device connector 134. However, their operation parameters (e.g., vacuum level, rotational or translation speed, etc.) may be different. In some embodiments, the vacuum applied to first working device 102 is lower (e.g., less than about 50 mm Hg), and the vacuum applied to second working device 102 is higher (e.g., greater than about 150 mmHg). Such operational parameters may be selected using one or more input elements 138. In such embodiments, a first working device 102 is introduced through a hysteroscope 100 and is used to perform a diagnostic or therapeutic procedure in the uterine cavity. The user may use one or more input elements 138 or other control mechanisms to select the working parameters of first working device 102. Thereafter, first working device 102 and hysteroscope 100 are removed from the patient. A second working device 102 may be introduced through the same or another hysteroscope 100 and used to perform a diagnostic or therapeutic procedure in the uterine cavity. The user may use one or more input elements 138 or other control mechanisms to select the working parameters of second working device 102. In some embodiments, first working device 102 is a debris removal device, and the second working device 102 is a resection device. In some method embodiments, the first working device 102 is a debris removal device, and the second working device 102 is an ablation device.

Endoscopes 100 described herein may be designed using a variety of structural and optical elements. In some embodiments, endoscopes 100 may be rigid endoscopes with a series of aligned glass rod lenses encased within a rigid, elongated insertion tube. High-resolution optical images are relayed from a distal objective lens to a proximal eyepiece or attached camera unit. In some embodiments, hysteroscopes 100 may be flexible fiberoptic endoscopes with bundled, coherent optical fibers. In some embodiments, endoscopes 100 may be digital endoscopes with a "chip-on-tip" design with one or more solid-state image sensors 704, such as a CMOS or CCD sensor(s) located on the distal region of the endoscope 100. Such digital endoscopes 100 may be rigid or flexible. Such digital endoscopes may capture and electronically transmit digital video signals to an external video processing and/or display system which may be located in a controller 130.

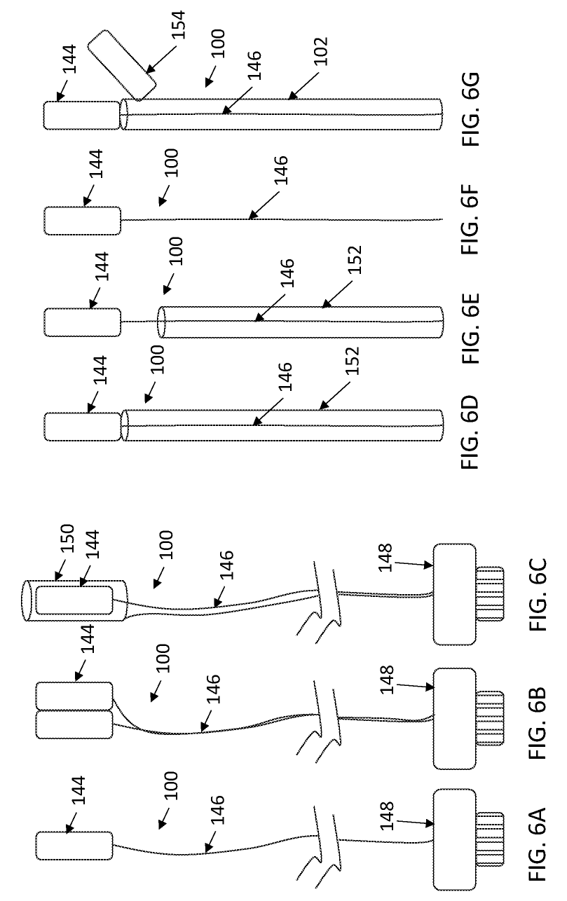

FIGS. 6A – 6C show examples of flexible digital endoscopes. In FIG. 6A, endoscope 100 comprises a distal sensor 144 (e.g., image sensor, CMOS sensor, CCD sensor, etc.) that is connected through one or more wires 146 to a proximal electrical connector 148. In FIG. 6B, endoscope 100 comprises multiple distal sensors 144 that are connected through one or more wires 146 to a proximal electrical connector 148. In FIG. 6C, endoscope 100 comprises one or more distal sensors 144 housed within a sensor casing 150 that are connected through one or more wires 146 to a proximal electrical connector 148. Sensor casing 150 further comprises one or more additional sensors (examples of which are disclosed herein) that are connected through one or more wires 146 to proximal electrical connector 148.

FIGS. 6D – 6G show the steps of an example of a method of using a flexible digital endoscope. In the step of FIG. 6D, a flexible endoscope 100 is introduced into the anatomy using a sheath 152. One or more wires 146 may be located inside sheath 152. Example designs of endoscope 100 are shown in FIGS. 6A – 6C. Distension fluid may be introduced through sheath 152. In the step of FIG. 6E, sheath 152 is withdrawn after placing the distal tip of endoscope 100 in an anatomical cavity. As shown in FIG. 6F, endoscope 100 is left behind in the anatomy. In the step of FIG. 6G, a working device 102 comprising a working element 154 is advanced over wires 146 into the anatomical cavity. Working device 102 may comprise one or more working elements 154 (examples of which are disclosed herein), which may be used to perform one or more diagnostic and/or therapeutic procedures under endoscopic guidance. Examples of working elements 154 include, but are not limited to: ablating elements, energy delivery elements, cutting elements, puncturing elements, substance delivery elements, etc.

### EXAMPLES

Example 1. A system for imaging an interior of an anatomical cavity, the system comprising: an endoscope configured for an insertion into the anatomical cavity; a display device; and a controller receiving an imaging output of the endoscope, wherein the controller comprises a processor and a memory storing instructions and one or more trained artificial intelligence models, wherein the instructions, when executed by the processor, cause the controller to: process the imaging output of the endoscope using the one or more trained artificial intelligence models to generate computed information of the anatomical cavity, wherein the computed information comprises one or more of: size information of the anatomical cavity, shape information of the anatomical cavity, and identification of pathology; and display a graphical overlay on the display device, the graphical overlay displaying the computed information of the anatomical cavity and the imaging output of the endoscope.

Example 2. The system of any one of the preceding examples, but particularly example 1, further comprising a working device configured for insertion into the anatomical cavity, wherein the instructions, when executed by the processor, cause the controller to track the working device inside the anatomical cavity.

Example 3. The system of any one of the preceding examples, but particularly example 1, further comprising means for a user to manually annotate one or more regions of the anatomical cavity.

Example 4. The system of any one of the preceding examples, but particularly example 1, wherein the system generates a two-dimensional representation of a portion of a wall of the anatomical cavity.

Example 5. The system of any one of the preceding examples, but particularly example 4, wherein the two-dimensional representation comprises one or more of: shape contour of the anatomical cavity, converted image of a three-dimensional region of the anatomical cavity, outline of a section through a plane of the anatomical cavity, etc.

Example 6. The system of any one of the preceding examples, but particularly example 4, wherein the two-dimensional representation is displayed on the display device.

Example 7. The system of any one of the preceding examples, but particularly example 1, wherein the system comprises a working device and the controller calculates one or more working parameters of the working device.

Example 8. A method of performing a diagnostic or therapeutic procedure on an anatomical cavity comprising: inserting an endoscope into the anatomical cavity; imaging a wall of the anatomical cavity using the endoscope; determining one or more contours of the wall of the anatomical cavity; and using the one or more contours to guide placement and/or function of working device and/or the endoscope within the anatomical cavity.

Example 9. The method of any one of the preceding examples, but particularly example 8, further comprising: guiding a user to image unimaged areas of the anatomical cavity.

Example 10. The method of any one of the preceding examples, but particularly example 8, further comprising: generating a two-dimensional representation of the anatomical cavity.

Example 11. The method of any one of the preceding examples, but particularly example 10, further comprising: displaying the two-dimensional representation and one or more system-generated annotations.

Example 12. The method of any one of the preceding examples, but particularly example 8, further comprising: tracking the working device in the anatomical cavity.

Example 13. The method of any one of the preceding examples, but particularly example 8, further comprising: determining anatomical size and shape information of the anatomical cavity.

Example 14. The method of any one of the preceding examples, but particularly example 8, further comprising generating a three-dimensional model of the anatomical cavity based on the one or more contours and a plurality of image frames obtained from the imaging of the wall of the anatomical cavity.

Example 15. The method of any one of the preceding examples, but particularly example 8, further comprising applying one or more trained artificial intelligence models to the imaging of the wall of the anatomical cavity to identify a pathology within the anatomical cavity.

Example 16. The method of any one of the preceding examples, but particularly example 15, further comprising determining one or more measurements of the pathology including one or more of size, shape, or spatial location of the pathology.

Example 17. The method of any one of the preceding examples, but particularly example 8, further comprising displaying one or more graphical overlays including one or more of alignment markers, trajectory indicators, or measurement annotations on a display device.

Example 18. The method of any one of the preceding examples, but particularly example 8, further comprising determining a position, orientation, or depth of the working device relative to one or more anatomical landmarks of the anatomical cavity.

Example 19. The method of any one of the preceding examples, but particularly example 8, further comprising generating one or more risk indicators associated with placement or operation of the working device within the anatomical cavity.

Example 20. The method of any one of the preceding examples, but particularly example 8, further comprising updating the one or more contours and corresponding guidance in real time based on the imaging of the wall of the anatomical cavity.

Although various inventive aspects have been described across multiple figures, various method embodiments are possible that combine multiple inventive aspects. For example, an endometrial ablation procedure may be performed using one or more of the following steps with ablation devices having an active end:

Pre-procedure hysteroscopy: Hysteroscope 100 may be navigated through cervix and uterine cavity. Example embodiments shown in FIGS. 1A – 1G and 1L-1M may be used in this step in this step.

Visualization of the uterine cavity: In this step, the uterine cavity is visualized and suitability of the uterus for endometrial ablation may be determined. The uterine cavity may be checked for weakened wall regions, perforations, false passages, etc. In this step, 3D mapping of the uterine cavity may be performed using any suitable method described in this specification. The width and/or length of the uterine cavity may be measured using any suitable method described in this specification. This may be used to determine one or more treatment parameters of the endometrial ablation procedure. Example embodiment shown in FIG. 1H may be used in this step.

Simulation of device deployment: In this step, the deployment of the active end (e.g. energy delivery elements, thermal elements, antennas) within the uterine cavity is simulated. Example embodiments shown in FIGS. 2I – 2L may be used in this step. Parameters that may be simulated include, but are not limited to: deployed width at the fundus, maximum possible deployed width in the uterine cavity, deployed length setting of the active end, position or output of width indicator dials or other displays, returned power or other electrical measures of active end deployment, etc.

Administering anesthesia: Local anesthesia may be injected and/or infused at desired locations. The system may guide the user regarding the sites of injection, number of injection sites, depth of injection, etc. Example embodiments shown in FIGS. 2G – 2H may be used in this step.

Determination of endometrial characteristics: In this step, qualitative (classification of endometrium into thick/medium/thin endometrium) and/or quantitative analysis of endometrium (e.g. estimates of endometrial thickness) is performed. This may be used to determine one or more treatment parameters of the endometrial ablation procedure.

Determination of device insertion depth: In this step, uterine sounding length may be calculated. This sounding length may be used during device insertion and positioning steps to ensure proper placement, avoid perforation, etc. Example embodiments shown in FIGS. 3N – 3O may be used in this step.

Calculation of energy delivery and other treatment parameters: One or more treatment parameters and positions of the active end (e.g. fundal position, proximal position, position of one or more lesion extending steps, etc.) may be determined. Examples of energy delivery parameters include, but are not limited to: power, energy delivery time, energy dose, delivery locations, cutoff such as returned power and impedance cutoffs, etc.

Generation of pre-procedure hysteroscopy report: Example embodiment shown in FIGS. 3P may be used in this step.

Device insertion: The endometrial ablation device may be inserted as per its instruction for use. The insertion depth may be compared with calculated or measured sounding length.

Device deployment: The active end of the endometrial ablation device may be deployed as per its instruction for use. One or more deployment parameters (e.g. deployed width and other mechanical measurements, returned power and other electrical measurements, etc.) may be checked with expected deployment parameters determined previously.

Energy delivery: The active end of the endometrial ablation device may be used for energy delivery to the endometrium as per its instruction for use. Feedback about the energy delivery may be obtained.

Post-procedure hysteroscopy: In this step, unablated or inadequately ablated regions may be determined. The endometrial ablation procedure may be repeated if necessary. The uterine cavity may be checked for perforation, false passages, etc.

Generation of post-procedure hysteroscopy report: Example embodiment shown in FIG. 3P may be used in this step.

Examples of endometrial ablation devices include, but are not limited to Novasure® system (described in US patents US6872183B2, US7407502B2, US6813520B2, etc.), Minitouch™ system (described in US patents US12144542B2, US11895760B2, and US11147619B2), Minerva® system (described in US patents US9289257B2, US8715278B2, and US8540708B2), and Cerene® system (described in US patents US9848933B2 and US9895183B2); the entire disclosures of each of which are incorporated herein by reference in their entireties.

In some embodiments, a device introduced through the channel of a hysteroscope 100 may be used as a guide to enter the cervical canal or navigate through the uterine cavity. Examples of such devices include, but are not limited to: guide wires, devices with atraumatic tips, devices with curved tips, devices with adjustable distal end projections, devices with adjustable stiffness, navigable device etc. The device may be steered using any of the guidance methods and devices disclosed herein. In other embodiments, the device may be introduced separately from the hysteroscope channel. References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “image” may include, and is contemplated to include, a plurality of images. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for imaging an interior of an anatomical cavity, the system comprising:
an endoscope configured for an insertion into the anatomical cavity;
a display device; and
a controller receiving an imaging output of the endoscope, wherein the controller comprises a processor and a memory storing instructions and one or more trained artificial intelligence models, wherein the instructions, when executed by the processor, cause the controller to:
process the imaging output of the endoscope using the one or more trained artificial intelligence models to generate computed information of the anatomical cavity, wherein the computed information comprises one or more of: size information of the anatomical cavity, shape information of the anatomical cavity, and identification of pathology; and
display a graphical overlay on the display device, the graphical overlay displaying the computed information of the anatomical cavity and the imaging output of the endoscope.

2. The system of claim 1, further comprising a working device configured for insertion into the anatomical cavity, wherein the instructions, when executed by the processor, cause the controller to track the working device inside the anatomical cavity.

3. The system of claim 1, further comprising means for a user to manually annotate one or more regions of the anatomical cavity.

4. The system of claim 1, wherein the system generates a two-dimensional representation of a portion of a wall of the anatomical cavity.

5. The system of claim 4, wherein the two-dimensional representation comprises one or more of: shape contour of the anatomical cavity, converted image of a three-dimensional region of the anatomical cavity, outline of a section through a plane of the anatomical cavity, etc.

6. The system of claim 4, wherein the two-dimensional representation is displayed on the display device.

7. The system of claim 1, wherein the system comprises a working device and the controller calculates one or more working parameters of the working device.

8. A method of performing a diagnostic or therapeutic procedure on an anatomical cavity comprising:
inserting an endoscope into the anatomical cavity;
imaging a wall of the anatomical cavity using the endoscope;
determining one or more contours of the wall of the anatomical cavity; and
using the one or more contours to guide placement and/or function of working device and/or the endoscope within the anatomical cavity.

9. The method of claim 8, further comprising: guiding a user to image unimaged areas of the anatomical cavity.

10. The method of claim 8, further comprising: generating a two-dimensional representation of the anatomical cavity.

11. The method of claim 10, further comprising: displaying the two-dimensional representation and one or more system-generated annotations.

12. The method of claim 8, further comprising: tracking the working device in the anatomical cavity.

13. The method of claim 8, further comprising: determining anatomical size and shape information of the anatomical cavity.

14. The method of claim 8, further comprising generating a three-dimensional model of the anatomical cavity based on the one or more contours and a plurality of image frames obtained from the imaging of the wall of the anatomical cavity.

15. The method of claim 8, further comprising applying one or more trained artificial intelligence models to the imaging of the wall of the anatomical cavity to identify a pathology within the anatomical cavity.

16. The method of claim 15, further comprising determining one or more measurements of the pathology including one or more of size, shape, or spatial location of the pathology.

17. The method of claim 8, further comprising displaying one or more graphical overlays including one or more of alignment markers, trajectory indicators, or measurement annotations on a display device.

18. The method of claim 8, further comprising determining a position, orientation, or depth of the working device relative to one or more anatomical landmarks of the anatomical cavity.

19. The method of claim 8, further comprising generating one or more risk indicators associated with placement or operation of the working device within the anatomical cavity.

20. The method of claim 8, further comprising updating the one or more contours and corresponding guidance in real time based on the imaging of the wall of the anatomical cavity.
